(19) United States
(12) Patent Application Publication
(10) Pub. No.: US 2012/0302534 A1
(43) Pub. Date: Nov. 29, 2012

(54) DERIVATIVES OF BETULIN

(76) Inventors: Daxin Gao, Pudong (CN); Nianhe Han, Pudong (CN); Zhimin Jin, Pudong (CN); Fangxian Ning, Pudong (CN); Jun Tang, Pudong (CN); Yongyong Wu, Pudong (CN); Heping Yang, Pudong (CN)

(21) Appl. No.: 13/577,452

(22) PCT Filed: Feb. 9, 2011
(86) PCT No.: PCT/US11/24174

§ 371 Date: Aug. 7, 2012

Related U.S. Application Data
(60) Provisional application No. 61/303,520, filed on Feb. 11, 2010.

(51) Int. Cl. A61K 31/568; A61P 31/18; A61K 31/58; C07J 53/00

(52) U.S. Cl. 514/176; 552/510; 514/182; 540/47; 514/172; 514/180

(57) ABSTRACT

The present invention relates to a compound characterized by the following Formula I:

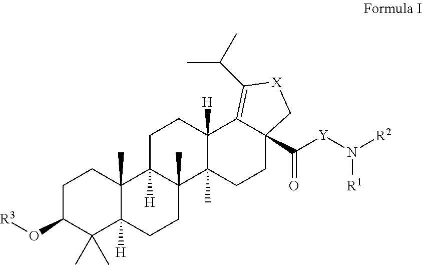

or a pharmaceutically acceptable salt thereof, wherein R¹, R², R³, X, and Y are as described herein. Compounds of the present invention are useful for the treatment of HIV-1.

### CROSS REFERENCE TO RELATED APPLICATIONS

This application is filed pursuant to 35 USC 371 as a United States National Phase Application of International Patent Application Serial No. PCT/US2011/024174 filed on Feb. 9, 2011, which claims priority from 61/303,520 filed on Feb. 11, 2010 in the United States.

### BACKGROUND OF THE INVENTION

Presently, long-term suppression of viral replication with antiretroviral drugs is the only option for treating HIV-1 infection. To date, a number of approved drugs have been shown to greatly increase patient survival. However, therapeutic regimens known as highly active antiretroviral therapy (HAART) are often complex because a combination of different drugs must be administered to the patient to avoid the rapid emergence of drug-resistant HIV-1 variants. Despite the positive impact of HAART on patient survival, drug resistance can still occur.

The emergence of multidrug-resistant (MDR) HIV-1 isolates has serious clinical consequences and must be suppressed with a new drug regimen, known as salvage therapy. Current guidelines recommend that salvage therapy includes at least two, and preferably three, fully active drugs. Typically, first-line therapies combine three to four drugs targeting the viral enzymes RT and protease (PR). One option for salvage therapy is to administer different combinations of drugs from the same mechanistic class that remain active against the resistant isolates. However, the options for this approach are often limited, as resistant mutations frequently confer broad cross-resistance to different drugs in the same class. Alternative therapeutic strategies have recently become available with the development of fusion, entry, and integrase (IN) inhibitors. However, resistance to all three new drug classes has already been reported both in vitro and in vivo. Sustained successful treatment of HIV-1-infected patients with antiretroviral drugs will therefore require the continued development of new and improved drugs with new targets and mechanisms of action.

The HIV Gag polyprotein precursor (Pr55Gag), which is composed of four protein domains—matrix (MA), capsid (CA), nucleocapsid (NC) and p6—and two spacer peptides, SP1 and SP2, represents a new therapeutic target. Although the cleavage of the Gag polyprotein plays a central role in the progression of infectious virus particle production, to date, no antiretroviral drug has been approved for this mechanism.

In most cell types, assembly occurs at the plasma membrane, and the MA domain of Gag mediates membrane binding. Assembly is completed by budding of the immature particle from the cell. Concomitant with particle release, the virally encoded PR cleaves Gag into the four mature protein domains, MA, CA, NC and p6, and the two spacer peptides, SP1 and SP2. Gag-Pol is also cleaved by PR, liberating the viral enzymes PR, RT and IN. Gag proteolytic processing induces a morphological rearrangement within the particle, known as maturation. Maturation converts the immature, donut-shaped particle to the mature virion, which contains a condensed conical core composed of a CA shell surrounding the viral RNA genome in a complex with NC and the viral enzymes RT and N. Maturation prepares the virus for infection of a new cell and is absolutely essential for particle infectivity.

Bevirimat (PA-457) is a maturation inhibitor that inhibits the final step in the processing of Gag, the conversion of capsid-SP1 (p25) to capsid, which is required for the formation of infectious viral particles. Bevirimat has activity against ART-resistant and wild-type HIV, and has shown synergy with antiretrovirals from all classes. Bevirimat reduced HIV viral load by a mean of 1.3 log₁₀/mL in patients who achieved trough levels of >=20 μg/mL and who did not have any of the key baseline Gag polymorphisms at Q369, V370 or T371. However, Bevirimat users with Gag polymorphisms at Q369, V370 or T371 demonstrated significantly lower load reductions than patients without Gag polymorphisms at these sites.

It would therefore be an advance in the art to discover alternative compounds that are maturation inhibitors.

### SUMMARY OF THE INVENTION

In a first aspect, the present invention is a compound characterized by the following formula:

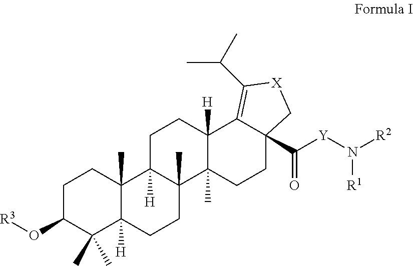

or a pharmaceutically acceptable salt thereof, wherein:

R¹ and R² are each independently H, C₁-C₆-alkyl, t-butyloxycarbonyl, Me-SO₂—, HOOCC(CH₃)₂CH₂C(O)—, CH₃C(O); (R⁴)₂N—(CH₂)ₘ—, (R⁵)ₙ-phenyl-Q-, (R⁶)_{q}-Hetaryl-(CH₂)ₚ—, (R⁶)_{q}-Hetalk-(CH₂)ᵣ—, or (R⁶)_{q}-Cycloalk-(CH₂)ₚ—; or R¹ and R², together with the nitrogen atom to which they are attached, form a 3- to 7-membered heterocycloalkyl ring optionally substituted with a methylsulfonyl group or up to two C₁-C₄-alkyl groups;

R³ is HOOCC(CH₃)₂CH₂C(O)— or HOOCCH₂C(CH₃)₂CH₂C(O)—;

each R⁴ is independently H or C₁-C₆-alkyl;

each R⁵ is independently halo, C₁-C₆-alkyl, C₁-C₆-alkoxy, CF₃, OCF₃, N(CH₃)₂, or NO₂;

each R⁶ is independently halo, C₁-C₆-alkyl, —COOH, —C(O)NH₂, dimethylaminomethyl, or 1-methyl-4-piperazinylmethyl,

X and Y are each independently methylene or carbonyl;

Q is —(CH₂)ₚ—, —C(O)—, —NH—C(O)—, —CH(CH₃)—, —C(CH₃)₂—, 1,1-cyclopropyldiyl, or 1,1-cyclopentyldiyl;

Hetaryl is a 5-6-membered heteroaryl group;

Hetalk is a 3-7-membered heterocycloalkyl group;

Cycloalk is a 3-6-membered cycloalkyl group;

each m is independently 2 or 3;

each n is independently 0, 1, or 2;

each p is independently 0 or 1;

each q is independently 0, 1, or 2; and

each r is independently 0, 1, 2, 3, or 4.

In a second aspect, the present invention relates to a composition comprising a) the compound of Formula I or a pharmaceutically acceptable salt thereof; and b) a pharmaceutically acceptable excipient.

In a third aspect, the present invention is a method of treating HIV-1 comprising administering to a patient suffering therefrom an effective amount of the compound of Formula I, or a pharmaceutically acceptable salt thereof.

Compounds of the present invention are useful for the treatment of patients with HIV-1.

### DETAILED DESCRIPTION OF THE INVENTION

The present invention relates to a compound of the following formula:

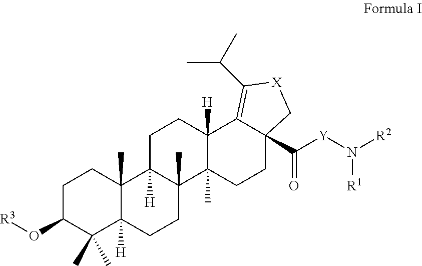

or a pharmaceutically acceptable salt thereof, where R¹, R², R³, X, and Y are as defined above.

As used herein, C₁-C₆-alkyl refers to a linear or branched alkyl group including methyl, ethyl, n-propyl, isopropyl, n-butyl, isobutyl, t-butyl, n-pentyl, and n-hexyl.

Halo is used herein to refer to fluoro, chloro, or bromo;

Hetaryl refers to a 5-6-membered heteroaryl group, examples of which include but not restricted to pyridyl, pyridazinyl, pyrimidinyl, pyrazinyl, oxazolyl, thiazolyl, imidazolyl, thienyl, furyl, and pyrrolyl.

Hetalk refers to a 3-7 membered heterocycloalkyl group, examples of which include azetidinyl, pyrrolidinyl, piperidinyl, N-methylpiperidin-4-yl, 4-methylpiperazinyl, morpholino, and thiomorpholino.

Cycloalk is used herein to mean cyclopropyl, cyclobutyl, cyclopentyl, and cyclohexyl.

R¹ and R², together with the nitrogen atom to which they are attached, may form a heterocycloalkyl group; examples of such groups include, but are not restricted to azetidinyl, piperidinyl, morpholino, thiomorpholino, piperazinyl, 4-methyl-piperazin-1-yl, 4-methylsulfonyl-piperazin-1-yl, 2,4-dimethyl-piperazin-1-yl, 4-methyl-diazepan-1-yl, 1-methyl-2-piperazinon-4-yl, thiomorpholine-1,1 dioxide-4-yl; and pyrrolidinyl groups.

In another aspect of the present invention, Hetaryl is pyridyl, thienyl, or furyl;

In another aspect, R¹ is H, methyl, dimethylaminoethyl, t-butyloxycarbonyl; Me-SO₂—, or HOOCC(CH₃)₂CH₂C(O)—; in another aspect, R¹ is H, CH₃, or dimethylaminoethyl;

In another aspect, R² is H, (R⁵)ₙ-phenyl-Q-, (R⁶)_{q}-furanyl-(CH₂)ₚ—, (R⁶)_{q}-pyridyl-(CH₂)ₚ—, (R⁶)_{q}-thienyl-(CH₂)ₚ—, 1-methylpyrazol-3-yl, Hetalk-(CH₂)ᵣ—, or C₃-C₆-cycloalkyl-(CH₂)ₚ—; in another aspect, R² is (R⁵)ₙ-phenyl-CH₂; thienyl-CH₂—; furanyl-CH₂; pyridinyl-CH₂—;

In another aspect R³ is HOOCC(CH₃)₂CH₂C(O)—;

In another aspect, each R⁴ is methyl and m is 2.

In another aspect, each R⁵ is independently methyl, methoxy, halo, CF₃, or OCF₃;

In another aspect, each R⁶ is independently methyl, F, or Cl.

In another aspect, X and Y are both carbonyl;

In another aspect, X and Y are both methylene;

In another aspect X is carbonyl and Y is methylene;

In another aspect X is methylene and X is carbonyl;

In another aspect, q is 0 or 1.

The present invention includes compounds as well as their pharmaceutically acceptable salts. Accordingly, the word “or” in the context of “a compound or a pharmaceutically acceptable salt thereof” is understood to refer to either a compound or a pharmaceutically acceptable salt thereof (alternative), or a compound and a pharmaceutically acceptable salt thereof (in combination).

The compounds of the present invention are derivatives of betulin referred to herein as Intermediate 1:

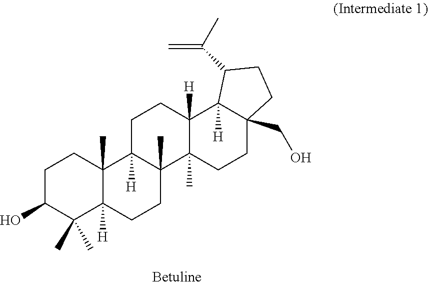

Betulin (lup-20(29)-ene-3β,28-diol) is an abundant naturally occurring triterpene commonly isolated from the bark of birch trees; betulin forms up to 30% of the dry weight of the extractive. See Green, Brian; Bentley, Michael D.; Chung, Bong Y.; Lynch, Nicholas G.; Jensen, Bruce L. (1985), “Isolation of Betulin and Rearrangement to Allobetulin A Biomimetic Natural Product Synthesis”, J. Chem. Educ. 200: 7.

As used herein, the term “pharmaceutically acceptable” refers to those compounds, materials, compositions, and dosage forms which are, within the scope of sound medical judgment, suitable for use in contact with the tissues of human beings and animals without excessive toxicity, irritation, or other problem or complication. The skilled artisan will appreciate that pharmaceutically acceptable salts of compounds according to formula (I) may be prepared. These pharmaceutically acceptable salts may be prepared in situ during the final isolation and purification of the compound, or by separately reacting the purified compound in its free acid or free base form with a suitable base or acid, respectively.

Compounds of the present invention can form pharmaceutically acceptable salts by reaction with a suitable acid or base. Suitable acids include inorganic and organic acids; examples of suitable inorganic acids include hydrochloric, hydrobromic, phosphoric, metaphosphoric, nitric, and sulfuric acids; examples of suitable organic acids include tartaric, acetic, trifluoroacetic, citric, malic, lactic, fumaric, benzoic, formic, propionic, glycolic, gluconic, maleic, succinic, methanesulfonic, ethanesulfonic, stearic, benzenesulfonic, bromobenzenesulfonic, and p-toluenesulfonic acids. Suitable bases include, for example, hydroxides, carbonates, hydrides, and alkoxides including NaOH, KOH, Na₂CO₃, K₂CO₃, NaH, and potassium-t-butoxide.

The embodiments of the invention described above are intended to be merely exemplary; numerous variations and modifications will be apparent to those skilled in the art. All such variations and modifications are intended to be within the scope of the present invention as defined in any appended claims.

### Equipment Description

¹H NMR spectra were recorded on a Bruker Avance-III 400 spectrometer. Chemical shifts are expressed in parts per million (ppm, δ units). Coupling constants are in units of hertz (Hz). Splitting patterns describe apparent multiplicities and are designated as s (singlet), d (doublet), t (triplet), q (quartet), quint (quintet), m (multiplet), br (broad).

The analytical low-resolution mass spectra (MS) were recorded on Agilent 1200 HPLC/6110 or Agilent 1200 HPLC/6130 using a SunFire C18, 4.6×50 mm, 3.5 μm using a gradient elution method.

Solvent A: 0.01% trifluoroacetic acid (TFA) in water;

Solvent B: 0.01% TFA in acetonitrile;

Constant A for 1.2 min followed by 5%-95% or 20%-95% B over 4 min.

### Biological Assay

The antiviral activity of test compounds was determined in a two cell co-culture HIV lifecycle assay. In this assay Jurkat T-lymphocytes that are chronically infected with HIV-1 HxB2 were co-cultured with indicator HOS cells that harbor a modified HIV LTR-luciferase reporter. Virus produced by the infected Jurkat cells can infect the HOS cells leading to LTR-directed expression of the luciferase reporter. Compounds that interfere with virus production in the Jurkat cells, maturation of the virus, entry or post-entry steps in the HIV lifecycle decrease the luciferase signal.

Prior to the beginning the assay, a frozen stock vial of infected Jurkat, J4HxB2, cells are rapidly thawed in a 37° C. waterbath and slowly diluted to 15 mL with cell medium (RPMI 1640 medium containing 10% fetal bovine serum and gentamycin) with gentle swirling. The cells are then placed into culture at 37° C., 5% CO₂, and expanded to 30 mL on day 4 and to 60 mL on day 7 by the addition of cell medium.

On the day the assay were started, HOS cells were rapidly thawed, slowly diluted to 15 mL in cell culture medium, centrifuged at 1400 rpm for 5 min and resuspended in 10 mL cell medium. 2×10⁷ HOS cells were diluted to 1112 mL in chilled (4° C.) cell medium and placed on a stir plate. 6.7×10⁷ J4HxB2 cells in culture were pelleted by centrifugation at 1400 rpm for 5 min and resuspended into the chilled HOS cell suspension. The HOS and J4HxB2 cells were mixed on the stir plate for at least 5 min prior to plating into 96- or 384-well plates. A Multidrop (or similar instrument) was used to dispense the cells into assay plates containing test compounds. In the 96-well assay format 0.2 mL J4HxB2/HOS cell suspension was added to 2 μL of test compound in the assay plate. For the 384-well assay format, 0.05 mL cell suspension was added to 0.5 μL of test compound in the assay plate. Compounds were tested as 10- or 11-point serial dilutions. After addition of cells to compound plates, plates were allowed to sit at room temperature for 30 min to 1 h then moved to humidified 5% CO₂ 37° C. incubator for 5 days. At the end of five days, plates were removed from the incubator and equilibrated to room temperature for 30 min to 1 h. Promega Steady-Glo reagent was prepared according to the manufacturer's directions and added to plates using Multidrop (or similar instrument). 0.02 mL of Steady-Glo was added to the wells of 384-well plate. For the 96-well plate, 0.1 mL of medium was removed from each well and 0.06 mL of Steady-Glo was added Luminescence was then detected using an Envision or Topcount Microplate Reader or similar instrument. For data analysis, high and low signal controls corresponding to wells containing DMSO or an HIV-1 inhibitor (efavirenz, brecanavir or similar antiviral), were used to normalize the data which was then fit to a non-linear regression model using a suitable data analysis package to determine IC₅₀ values.

### Schemes and Experimental Procedures

The following schemes and procedures illustrate how compounds of the present invention can be prepared. The specific solvents and reaction conditions referred to are also illustrative and are not intended to be limiting. Compounds not described are either commercially available or are readily prepared by one skilled in the art using available starting materials. The Examples disclosed herein are for illustrative purposes only and are not intended to limit the scope of the invention. All examples exhibited LHIV IC₅₀ values between 1 μM and 1 nM using the assay disclosed herein.

Synthesis of Intermediate 10 for Formula I Compounds:

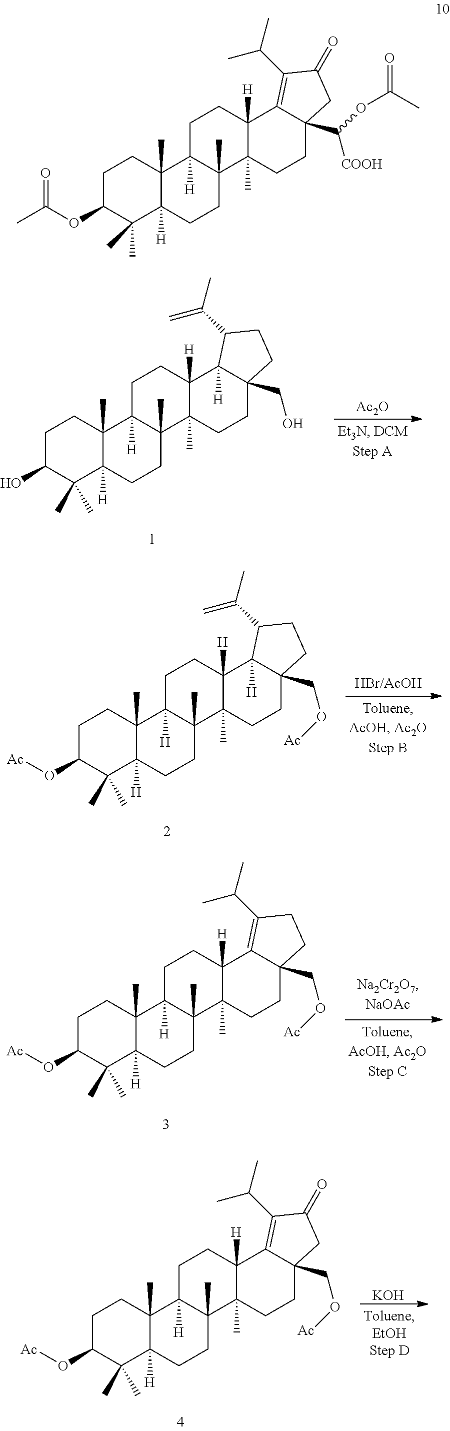

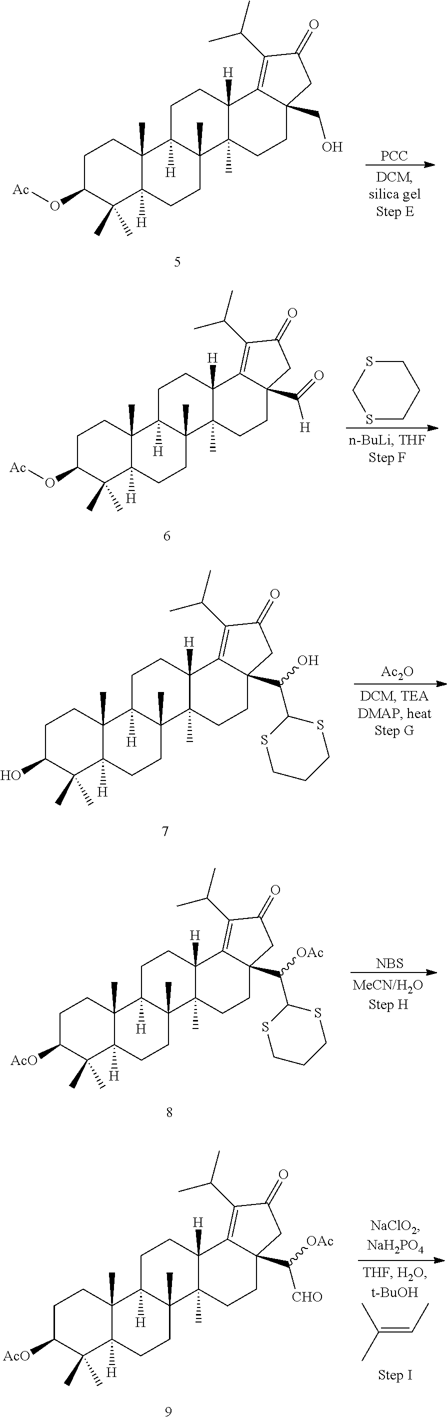

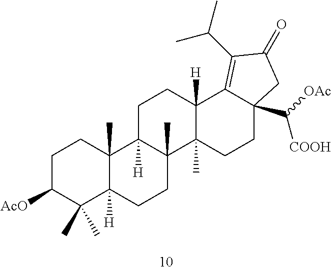

### Step A: Intermediate 2

To a solution of the intermediate 1 (20 g, 45.2 mmol), 4-dimethylaminopyridine (DMAP, 1.66 g, 13.6 mmol), and Et₃N (63 mL, 136 mmol) in CH₂Cl₂ (DCM, 100 mL) at room temperature was added acetic anhydride (Ac₂O, 17.1 mL, 113 mmol). After it was heated at reflux overnight, and cooled down to room temperature, the reaction was quenched with water (50 mL). The organic phase was then washed with water (50 mL×2) and dried over sodium sulfate. After removing most of the organic solvent under reduced pressure, anhydrous ethanol (50 mL) was added and the resulting precipitates were collected by filtration as a white solid (intermediate 2, 20 g, 84%). ¹H NMR (400 MHz, CDCl₃) δ ppm 4.69 (1H, m), 4.59 (1H, m), 4.51-4.43 (1H, m), 4.25 (1H, d, J=11.2 Hz), 3.85 (1H, d, J=10.8 Hz), 2.49-2.40 (1H, m), 2.07 (3H, s), 2.04 (3H, s), 1.98-0.77 (42H, m). LC/MS: m/z calculated 526.4, found 527.7 (M+1)+.

### Step B: Intermediate 3

HBr in acetic acid (40 mL, 33%) was added to a suspension of the intermediate 2 (20 g, 38 mmol) in toluene (40 mL), Ac₂O (40 mL), and acetic acid (AcOH, 40 mL) previously heated at 105° C. The reaction mixture was stirred and heated at this temperature for 1.5 h. After cooling down, sodium acetate (24 g) was added and the resulting reaction mixture was evaporated to dryness. The pale brownish residue was taken up in DCM (200 mL) and the organic phase was washed with water (100 mL×3), dried over sodium sulfate, and evaporated to dryness under reduced pressure to give a residue, which was recrystallized from 95% ethanol and DCM to give the intermediate 3 (13.8 g, 69%) as a white solid. ¹H NMR (400 MHz, CDCl₃) δ ppm 4.50-4.46 (1H, m), 4.02 (1H, d, J=10.8 Hz), 3.98 (1H, d, J=10.8 Hz), 3.18-3.10 (1H, m), 2.43-2.40 (1H, m), 2.26-2.22 (2H, m), 2.04 (3H, s), 2.05 (3H, s), 2.00-1.95 (1H, m), 1.90-1.85 (1H, m), 1.77-0.83 (39H, m). LC/MS: m/z calculated 526.4, found 549.2 (M+Na)+.

### Step C: Intermediate 4

A mixture of the intermediate 3 (7 g, 13.29 mmol), sodium acetate (NaOAc, 6.21 g, 76 mmol) and sodium dichromate dihydrate (4.75 g, 15.95 mmol) in anhydrous toluene (90 mL), AcOH (119 mL), and Ac₂O (29 mL) was stirred at 60° C. overnight. After cooling down, the reaction mixture was partitioned between water (150 mL) and ethyl acetate (EtOAc, 250 mL). The organic phase was washed successively with water (100 mL), saturated solution of sodium carbonate (100 mL×2) and brine (100 mL×2), dried over sodium sulfate, and concentrated under reduced pressure to afford a sticky oil. The sticky oil was triturated with methanol (MeOH, 250 mL) and the precipitates were collected as the intermediate 4 (6 g, 11.1 mmol, 83% yield) as a white solid. ¹H NMR (400 MHz, CDCl₃) δ ppm 4.52-4.46 (1H, m), 4.33 (1H, d, J=10.8 Hz), 4.06 (1H, d, J=11.2 Hz), 3.21-3.16 (1H, m), 2.86 (1H, dd, J=12.8, 3.2 Hz), 2.42-2.36 (1H, m), 2.05 (3H, s), 2.00 (3H, s), 1.94-0.84 (40H, m). LC/MS: m/z calculated 540.4, found 563.3 (M+Na)⁺.

### Step D: Intermediate 5

A mixture of the intermediate 4 (7 g, 12.94 mmol) and potassium hydroxide (KOH, 0.872 g, 15.5 mmol) in a mixture of 1:1 ethanol (EtOH) and toluene (400 mL) was stirred vigorously at room temperature for 1 h. The reaction mixture was neutralized with aqueous HCl (1N) to pH=7 and evaporated to dryness. The obtained residue was taken up in water and a small amount of acetone. The precipitates were collected and then washed with water and dried in vacuo to obtain the intermediate 5 (6.0 g, 93%) as a white solid. LC/MS: m/z calculated 498.4, found 499.3 (M+1)+.

### Step E: Intermediate 6

To a solution of the intermediate 5 (5.1 g, 10.23 mmol) in DCM (300 mL) at room temperature were added pyridinium chlorochromate (PCC, 6.61 g, 30.7 mmol), and silica gel (6.6 g). The reaction mixture was stirred at room temperature for 1 h. After the reaction was quenched with water, the organic phase was washed with saturated sodium bicarbonate solution (100 mL), dried over sodium sulfate, and evaporated under reduced pressure to give a crude product, which was purified by column chromatography on silica gel (EtOAc:PE=1:10 to 1:5) to give the intermediate 6 (4.2 g, 83%) as a white solid. LC/MS: m/z calculated 496.4, found 497.2 (M+1)+.

### Step F: Intermediate 7

To a solution of 1,3-dithiane (5.7 g, 47.4 mmol) in anhydrous tetrahydrofuran (THF, 60 mL) under an atmosphere of nitrogen at −40° C. was slowly added a solution of n-BuLi (27 mL, 67.5 mmol). After the reaction mixture was stirred at −20° C. for another 2 h, a solution of the intermediate 6 (4.2 g, 8.46 mmol) in anhydrous THF (40 mL) was slowly added under an atmosphere of nitrogen at −70° C. The reaction was then stirred at −78° C. for 1 h before it was quenched with a saturated solution of NaHCO₃. Extraction was conducted with EtOAc and the organic phase was washed with water (50 mL), saturated brine (50 mL), dried over sodium sulfate, and evaporated under reduced pressure to give a crude product, which was purified by column chromatography on silica gel (PE:EtOAc=8:1 to 4:1) to afford the intermediate 7 (3.0 g, 5.22 mmol, 61.7%). LC/MS: m/z calculated 574.4, found 575.0 (M+1)+.

### Step G: Intermediate 8

To a solution of the intermediate 7 (3.5 g, 6.09 mmol), Et₃N (2.55 mL, 18.26 mmol), and DMAP (0.149 g, 1.218 mmol) in DCM (40 mL) was added Ac₂O (3.45 mL, 36.5 mmol) at room temperature. After stirring at 50° C. for 2 h, the reaction was quenched with water. The organic phase was washed with water (100 mL), dried over sodium sulfate, and evaporated under reduced pressure to give the intermediate 8 (3.41 g, 85%). LC/MS: m/z calculated 658.4, found 659.1 (M+1)+.

### Step H: Intermediate 9

To a solution of the intermediate 8 (4.5 g, 6.83 mmol) in acetonitrile (160 mL) and water (40 mL) was added N-bromosuccinimide (NBS, 7.29 g, 41.0 mmol) at room temperature. After stirring at room temperature for 10 min, the reaction was quenched with water. The organic layer was washed with saturated sodium sulfite solution (200 mL), dried over sodium sulfate, and evaporated under reduced pressure to give the intermediate 9 (3.2 g, 82%). LC/MS: m/z calculated 568.4, found 569.3 (M+1)+.

### Step I: Intermediate 10

To an ice-cooled solution of the intermediate 9 (3.2 g, 5.63 mmol) in t-butanol (300 mL), THF (60 mL), and 2-methyl-2-butene (2 mL) was added slowly a solution of NaClO₂ (7.19 g, 67.5 mmol) and NaH₂PO₄ (6.75 g, 56.3 mmol) in water (60 mL) over 15 min. After stirring at 0° C. for 10 min, the reaction mixture was warmed to room temperature and stirred for another 30 min followed by the dilution with EtOAc. The organic phase was washed with water (100 mL), dried over sodium sulfate, filtered, and concentrated to dryness. The obtained residue was purified by column chromatography on silica gel (DCM:MeOH=50:1 to 10:1) to afford the intermediate 10 (2.8 g, 81%) as a white solid. LC/MS: m/z calculated 584.4, found 585.3 (M+1)+.

The following examples are for illustrative purposes only and not intended to limit the scope of the invention. For the scheme following Example 1, R¹NHR² represents an azetidinyl group; and R³ is HOOCC(CH₃)₂CH₂C(O)O—.

### Example 1

### 4-{[(3aR,5aR,5bR,7aR,9S,11aR,11bR,13aS)-3a-[1-Azetidinyl(oxo)acetyl]-5a,5b,8,8,11a-pentamethyl-1-(1-methylethyl)-2-oxo-3,3a,4,5,5a,5b,6,7,7a,8,9,10,11,11a,11b,12,13,13a-octadecahydro-2H-cyclopenta[a]chrysen-9-yl]oxy}-2,2-dimethyl-4-oxobutanoic acid

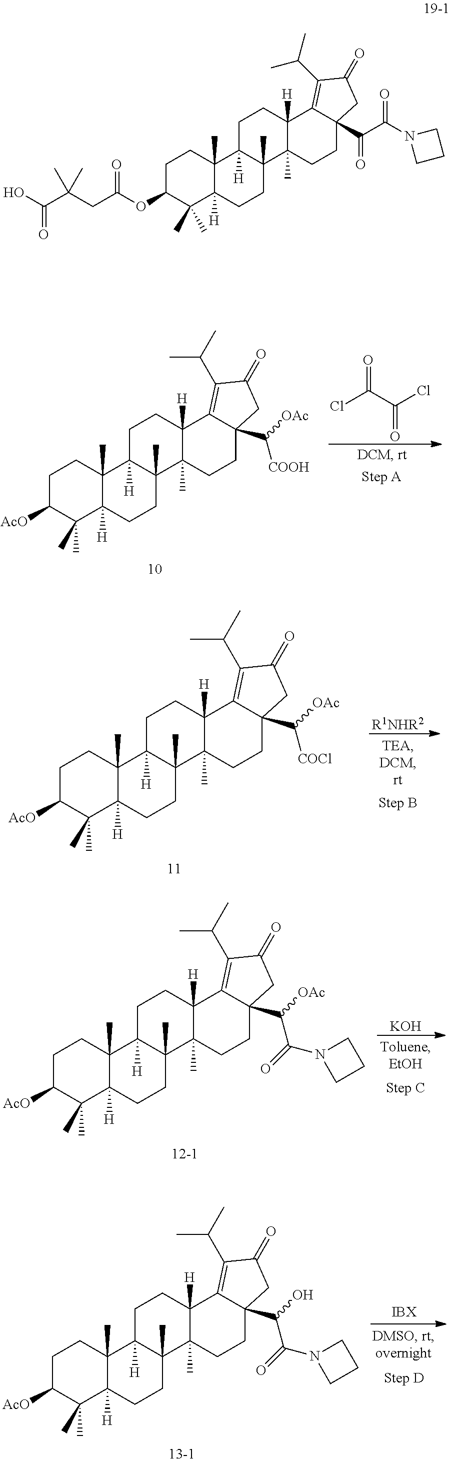

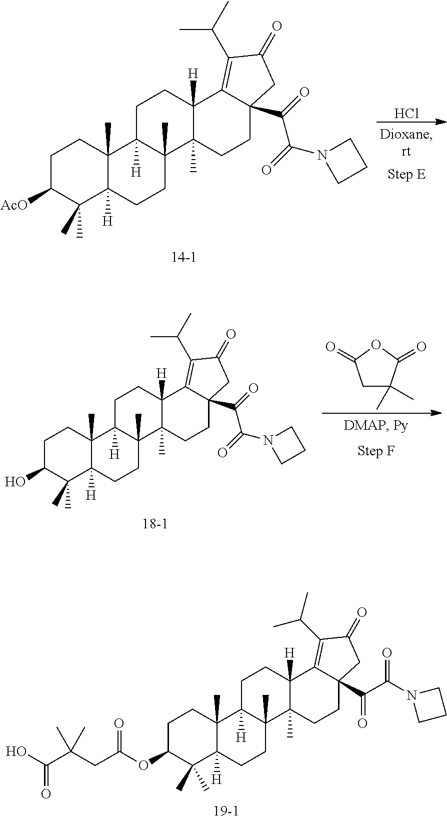

### Step A: Intermediate 11

To a solution of the intermediate 10 (680 mg, 1.163 mmol) in DCM (5 mL) were added oxalyl chloride (3 mL, 35.5 mmol) and a few drops of DMF. The reaction mixture was stirred at room temperature for 3 h and evaporated under reduced pressure to yield the intermediate 11 as a light yellow solid.

### Step B: Intermediate 12-1

To a solution of the intermediate 11 (430 mg, 0.713 mmol), DMAP (100.0 mg, 0.819 mmol) and Et₃N (2 mL, 14.35 mmol) in DCM (10 mL) was added azetidine (200 mg, 3.5 mmol) at room temperature. After stirring at room temperature for 4 h, the reaction was diluted with DCM (50 mL). The organic phase was washed with water (50 mL), aqueous hydrochloric acid (2 N, 50 mL), then dried over sodium sulfate, and evaporated under reduced pressure to give a crude product, which was purified by column chromatography on silica gel (PE:EtOAc=5:1 to 1:1) to afford the intermediate 12-1 (300 mg, 67.5%) as a white solid. LC/MS: m/z calculated 623.4, found 624.3 (M+1)+.

### Step C: Intermediate 13-1

A mixture of the intermediate 12-1 (350 mg, 0.561 mmol) and KOH (19 mg, 0.353 mmol) in a mixture of 1:1 EtOH and toluene (12 mL) was stirred vigorously at room temperature for 15 min. The reaction mixture was neutralized with aqueous HCl (1 N) to pH=7. After the addition of water, the reaction was extracted with EtOAc. The organic phase was dried over sodium sulfate, filtered, and concentrated under reduced pressure to give the intermediate 13-1 (320 mg, 97%) as a light yellow solid. LC/MS: m/z calculated 581.4, found 582.3 (M+1)+.

### Step D: Intermediate 14-1

To a solution of the intermediate 13-1 (430 mg, 0.74 mmol) in dimethylsulfoxide (DMSO, 8 mL) was added 2-iodoxybenzoic acid (IBX, 1.1 g, 3.93 mmol) at room temperature. After stirring at room temperature overnight, the reaction was quenched with water and extracted with EtOAc. The organic phase was dried over sodium sulfate, filtered, and concentrated under reduced pressure to give a residue, which was purified by column chromatography on silica gel (PE:EA=5:1 to 3:1) to afford the intermediate 14-1 (300 mg, 70%) as a white solid. LC/MS: m/z calculated 579.4, found 580.3 (M+1)+.

### Step E: Intermediate 18-1

A solution of the intermediate 14-1 (300 mg, 0.52 mmol) in 1,4-dioxane (15 mL) and concentrated HCl (5 mL) was stirred at room temperature overnight. The reaction was quenched with water (30 mL) and extracted with EtOAc (60 mL). The organic phase was washed with water, dried over sodium sulfate, and evaporated under reduced pressure to give a residue, which was purified by column chromatography on silica gel (PE:EtOAc=5:1 to 3:1) to afford the intermediate 18-1 (70 mg, 25%) as a white solid. LC/MS: m/z calculated 537.4, found 538.3 (M+1)+.

### Step F: Compound 19-1

To a solution of the intermediate 18-1 (70 mg, 0.13 mmol) in pyridine (3 mL) were added 3,3-dimethyldihydro-2,5-furandione (159 mg, 1.302 mmol) and DMAP (334 mg, 2.6 mmol). After it was heated at 90° C. overnight, the reaction mixture was extracted with DCM. The organic phase was washed with HCl (2 N, 25 mL), water (50 mL×2), dried over sodium sulfate, and evaporated under reduced pressure to give a residue, which was purified by column chromatography on silica gel (PE:EtOAc=3:1 to 2:1) to give the compound 19-1 (30 mg, 34.6%) as a white solid product. ¹H NMR (400 MHz, CDCl₃) δ ppm 4.54-4.42 (2H, m), 4.28-4.35 (1H, m), 4.05-4.15 (2H, m), 3.17-3.25 (1H, m), 2.75-2.62 (4H, m), 2.60-2.52 (2H, m), 2.35 (1H, quint, J=8.0 Hz), 2.17 (1H, d, J=19.2 Hz), 2.10-2.00 (1H, m), 1.96-1.82 (1H, m), 1.78-0.78 (42H, m). LC/MS: m/z calculated 665.9, found 664.4 (M−1)−.

### Example 2

### 2,2-Dimethyl-4-oxo-4-({(3aR,5aR,5bR,7aR,9S,11aR,11bR,13aS)-5a,5b,8,8,11a-pentamethyl-1-(1-methylethyl)-3a-[[4-(methylsulfonyl)-1-piperazinyl](oxo)acetyl]-2-oxo-3,3a,4,5,5a,5b,6,7,7a,8,9,10,11,11a,11b,12,13,13a-octadecahydro-2H-cyclopenta[a]chrysen-9-yl}oxy)butanoic acid

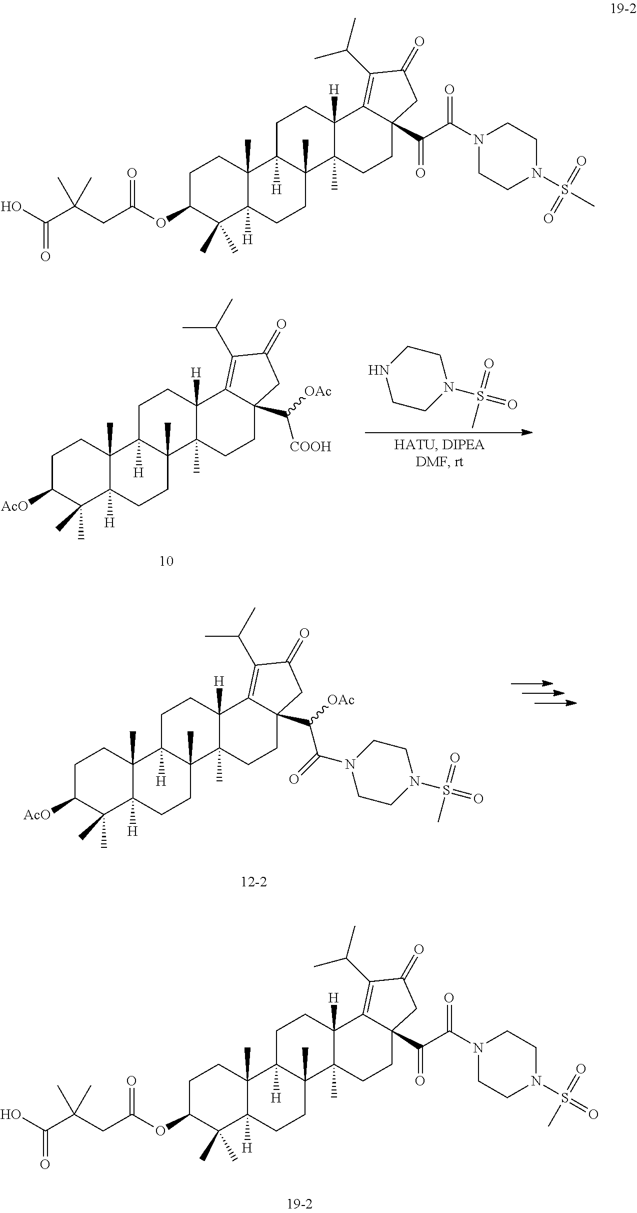

### Intermediate 12-2

To a mixture of the intermediate 10 (600 mg, 1.026 mmol), diisopropylethylamine (DIPEA, 2 g, 15.47 mmol), and 2-(1H-7-azabenzotriazol-1-yl)-1,1,3,3-tetramethyl uronium hexafluorophosphate methanaminium (HATU, 1.4 g, 3.68 mmol) in DMF (8 mL) was added 1-(methylsulfonyl)piperazine (700 mg, 4.26 mmol) at room temperature. After stirring at room temperature for 4 h, the reaction was quenched with water (100 mL), and extracted with EtOAc (200 mL). The organic phase was dried over sodium sulfate, and evaporated under reduced pressure to give a residue, which was purified by column chromatography on silica gel (DCM:MeOH=100:1 to 30:1) to afford the intermediate 12-2 (650 mg, 87%) as a white solid. LC/MS: m/z calculated 730.4, found 731.3 (M+1)+.

### Compound 19-2

Compound 19-2 was prepared as a white solid from the intermediate 12-2 with a procedure similar to that used in Example 1, where R¹NHR² is methylsulfonylpiperazine. ¹H NMR (400 MHz, CDCl₃) δ ppm 4.54-4.46 (1H, m), 3.82-3.73 (1H, m), 3.68-3.53 (2H, m), 3.50-3.42 (1H, m), 3.36-3.18 (5H, m), 2.82 (3H, s), 2.78-2.50 (5H, m), 2.20 (1H, d, J=18.4 Hz), 2.10-2.06 (1H, m), 1.90-0.82 (43H, m). LC/MS: m/z calculated 772.4, found 773.3 (M+1)+.

### Example 3

### 2,2-dimethyl-4-oxo-4-({(3aR,5aR,5bR,7aR,9S,11aR,11bR,13aS)-5a,5b,8,8,11a-pentamethyl-1-(1-methylethyl)-3a-[(4-methyl-1-piperazinyl)(oxo)acetyl]-2-oxo-3,3a,4,5,5a,5b,6,7,7a,8,9,10,11,11a,11b,12,13,13a-octadecahydro-2H-cyclopenta[a]chrysen-9-yl}oxy)butanoic acid

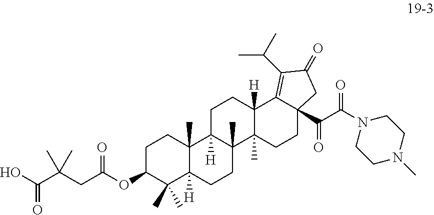

A similar procedure used to prepare the compound of Example 2 was followed to prepare the title compound as a light yellow solid. In this case, R¹NHR² is 1-methylpiperazine. ¹H NMR (400 MHz, DMSO-d⁶) δ ppm 4.38-4.32 (1H, m), 3.52-3.17 (6H, m), 2.60-2.20 (9H, m), 2.19 (3H, s), 1.98-0.79 (44H, m). LC/MS: m/z calculated 709.0, found 707.5 (M−1)−.

### Example 4

### 4-{[(3aR,5aR,5bR,7aR,9S,11aR,11bR,13aS)-3a-[[(4-Fluorophenyl)amino](oxo)acetyl]-5a,5b,8,8,11a-pentamethyl-1-(1-methylethyl)-2-oxo-3,3a,4,5,5a,5b,6,7,7a,8,9,10,11,11a,11b,12,13,13a-octadecahydro-2H-cyclopenta[a]chrysen-9-yl]oxy}-2,2-dimethyl-4-oxobutanoic acid

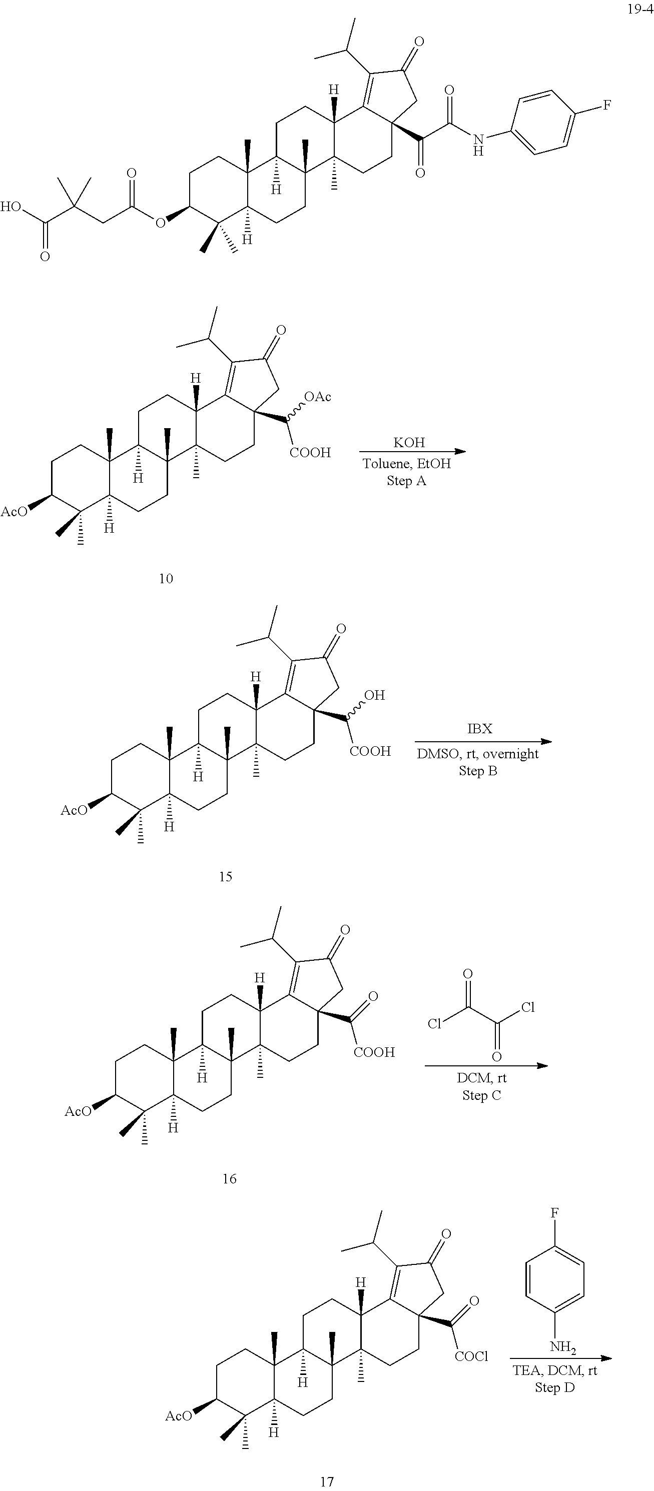

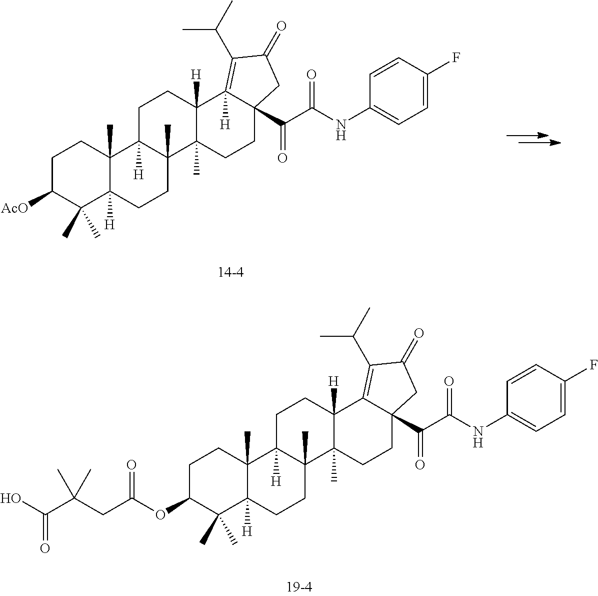

### Step A: Intermediate 15

To a solution of the intermediate 10 (500 mg, 0.855 mmol) in EtOH (2 mL) and toluene (2 mL) at room temperature was added KOH (192 mg, 3.42 mmol). After stirring at room temperature for 30 min, the reaction was diluted with DCM (100 mL). The organic phase was washed with aqueous NH₄Cl (50 mL×2) and brine (50 mL), dried over sodium sulfate and evaporated under reduced pressure to give the intermediate 15 (400 mg, 86%). LC/MS: m/z calculated 542.4, found 543.1 (M+1)+.

### Step B: Intermediate 16

To a solution of the intermediate 15 (290 mg, 0.534 mmol) in DMSO (6 mL) at room temperature was added IBX (1496 mg, 5.34 mmol). After it was stirred at 50° C. for 3 h, the reaction mixture was then cooled down to room temperature and diluted with DCM (100 mL). The organic phase was washed with water (50 mL×4), dried over sodium sulfate and evaporated under reduced pressure to give the intermediate 16 (234 mg, 81%) as a yellow solid. LC/MS: m/z calculated 540.4, found 541.1 (M+1)+.

### Step C: Intermediate 17

To a solution of the intermediate 16 (350 mg, 0.664 mmol) in DCM (8 mL) under an atmosphere of nitrogen at 0° C. was added slowly oxalyl chloride (1.4 mL, 15.99 mmol) over 5 min. After it was stirred at room temperature for 1 h, the reaction mixture was evaporated to dryness to give the intermediate 17 as a light yellow solid.

### Step D: Intermediate 14-4

A mixture of 4-fluoroaniline (221 mg, 1.992 mmol) and Et₃N (0.463 mL, 3.32 mmol) in DCM (7 mL) was added the intermediate 17 (371 mg, 0.664 mmol) at room temperature. After stirring at room temperature for 1 h, the reaction mixture was diluted with DCM (50 mL). The organic phase was washed with water (50 mL), brine (25 mL), dried over sodium sulfate, and evaporated under reduced pressure to give a residue, which was purified by column chromatography on silica gel (PE:EtOAc=5:1 to 1:1) to afford the intermediate 14-4 (150 mg, 36%) as a white solid. LC/MS: m/z calculated 634.4, found 635.3 (M+1)+.

### Compound 19-4

The compound 19-4 was prepared as a yellow solid from the intermediate 14-4 with a similar procedure used in Example 1. ¹H NMR (400 MHz, CDCl₃) δ ppm 8.72 (1H, s), 7.61-7.57 (2H, m), 7.08-7.03 (2H, m), 4.52-4.48 (1H, m), 3.28-3.20 (1H, m), 2.78-2.53 (5H, m), 2.25 (1H, d, J=18.8 Hz), 2.08-1.87 (2H, m), 1.18-0.81 (42H, m). LC/MS: m/z calculated 719.4, found 720.3 (M+1)+.

### Example 5

### 2,2-dimethyl-4-oxo-4-[((3aR,5aR,5bR,7aR,9S,11aR,11bR,13aS)-5a,5b,8,8,11a-pentamethyl-1-(1-methylethyl)-2-oxo-3a-{oxo[(2-thienylmethyl)amino]acetyl}-3,3a,4,5,5a,5b,6,7,7a,8,9,10,11,11a,11b,12,13,13a-octadecahydro-2H-cyclopenta[a]chrysen-9-yl)oxy]butanoic acid

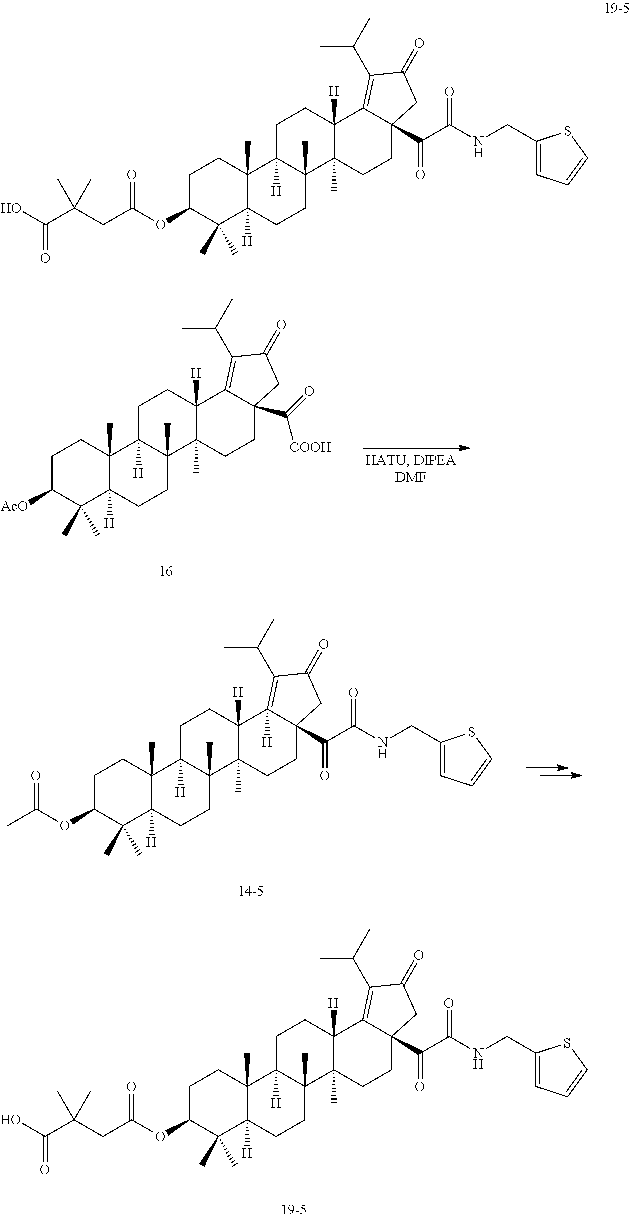

### Intermediate 14-5

To a solution of the intermediate 16 (220 mg, 0.407 mmol), HATU (309 mg, 0.814 mmol), and DIPEA (0.36 mL, 2.1 mmol) in DMF (10 mL) was added (2-thienylmethyl)amine (46 mg, 0.407 mmol) at room temperature. The reaction mixture was stirred at room temperature overnight and diluted with DCM. The organic phase was washed with water (50 mL), dried over sodium sulfate and evaporated to dryness in vacuo to give a residue, which was purified by column chromatography on silica gel (PE:EtOAc=20:1 to 4:1) to afford the intermediate 14-5 (60 mg, 23%) as a yellow solid. LC/MS: m/z calculated 636.4, found 637.3 (M+1)+.

### Compound 19-5

The compound 19-5 was prepared as a yellowish solid from the intermediate 14-5 with a similar procedure used in Example 1. ¹H NMR (400 MHz, CDCl₃): δ ppm 7.25 (1H, dd, J=4.8 Hz, 1.6 Hz), 7.19 (1H, t, J=5.6 Hz), 7.00-6.94 (2H, m), 4.66 (1H, dd, J=15.2 Hz, 6.0 Hz), 4.57 (1H, dd, J=15.2 Hz, 6.0 Hz), 4.54-4.47 (1H, m), 3.25-3.20 (1H, m), 2.76-2.52 (5H, m), 2.21 (1H, d, J=18.8 Hz), 2.08-1.86 (2H, m), 1.78-0.81 (42H, m). LC/MS: m/z calculated 722.4, found 723.3 (M+1)+.

### Example 6

### 4-{[(3aR,5aR,5bR,7aR,9S,11aR,11bR,13aS)-3a-[{[(4-Chlorophenyl)methyl]amino}(oxo)acetyl]-5a,5b,8,8,11a-pentamethyl-1-(1-methylethyl)-2-oxo-3,3a,4,5,5a,5b,6,7,7a,8,9,10,11,11a,11b,12,13,13a-octadecahydro-2H-cyclopenta[a]chrysen-9-yl]oxy}-2,2-dimethyl-4-oxobutanoic acid

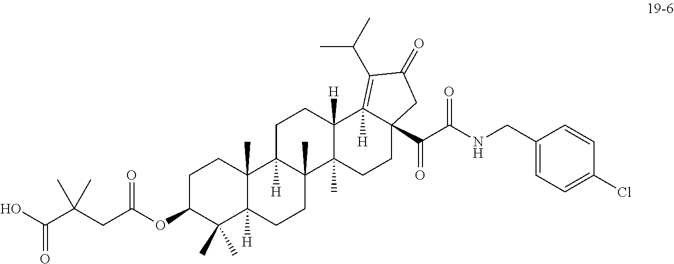

A similar procedure as described in Example 2, where R¹NHR² is [(4-chlorophenyl)methyl]amine, was used to prepare the title compound as a light yellow solid. ¹H NMR (400 MHz, CDCl₃) δ ppm 7.32-7.17 (5H, m), 4.54-4.32 (3H, m), 3.26-3.16 (1H, m), 2.76-2.48 (5H, m), 2.20 (1H, d, J=18.8 Hz), 2.03-1.86 (2H, m), 1.77-0.81 (43H, m). LC/MS: m/z calculated 749.4, found 748.4 (M−1)−.

### Example 7

### 4-{[(3aR,5aR,5bR,7aR,9S,11aR,11bR,13aS)-3a-[{[(2-chlorophenyl)methyl]amino}(oxo)acetyl]-5a,5b,8,8,11a-pentamethyl-1-(1-methylethyl)-2-oxo-3,3a,4,5,5a,5b,6,7,7a,8,9,10,11,11a,11b,12,13,13a-octadecahydro-2H-cyclopenta[a]chrysen-9-yl]oxy}-2,2-dimethyl-4-oxobutanoic acid

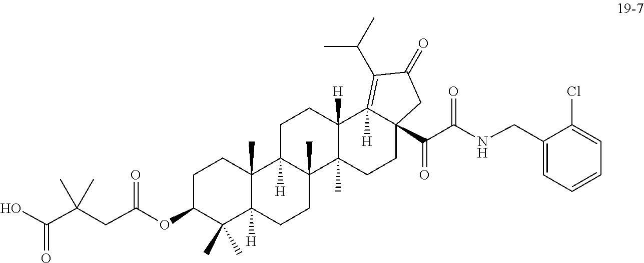

A similar procedure as described in Example 2, where R¹NHR² is [(2-chlorophenyl)methyl]amine, was used to prepare the title compound as a light yellow solid. ¹H NMR (400 MHz, CDCl₃): δ ppm 7.41-7.21 (5H, m), 4.61-4.44 (3H, m), 3.22-3.12 (1H, m), 2.76-2.46 (5H, m), 2.18 (1H, d, J=19.2 Hz), 1.96-1.84 (2H, m), 1.78-0.81 (43H, m). LC/MS: m/z calculated 749.4, found 748.4 (M−1)−.

The following compounds listed in Table 1 were prepared using methods similar to those listed above. In each case, X and Y are each carbonyl.

TABLE 1

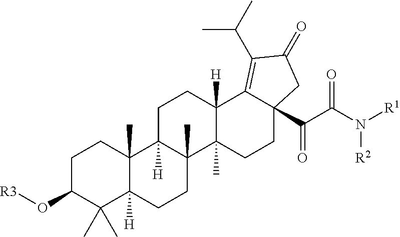

| Example | | | | |
|---|---|---|---|---|
| No. | R¹ | R² | R³ | Procedure |
| 7-1 | H | 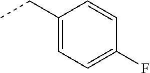 | 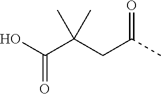 | Ex 2 |
| 7-2 | H | 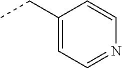 | 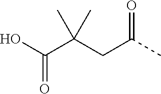 | Ex 2 |
| 7-3 | H | 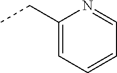 | 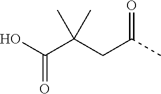 | Ex 2 |
| 7-4 | H | 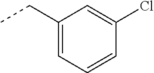 | 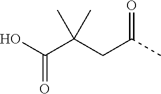 | Ex 2 |
| 7-5 | H | 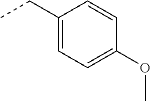 | 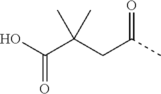 | Ex 2 |
| 7-6 | H | 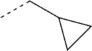 | 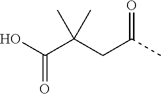 | Ex 2 |
| 7-7 | H | 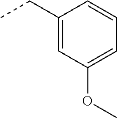 | 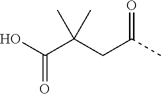 | Ex 2 |
| 7-8 | H | 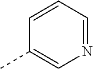 | 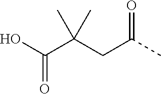 | Ex 1 |
| 7-9 | H | 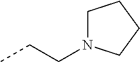 | 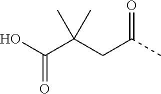 | Ex 1 |
| 7-10 | H | 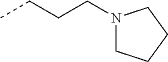 | 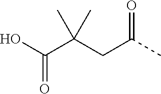 | Ex 1 |
| 7-11 | H | 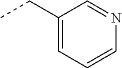 | 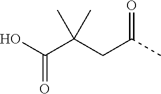 | Ex 2 |
| 7-12 | H | 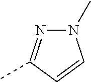 | 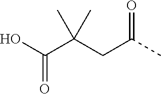 | Ex 5 |
| 7-13 | CH₃ | 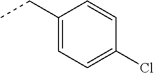 | 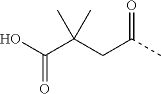 | Ex 2 |
| 7-14 | H | 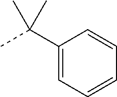 | 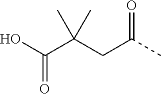 | Ex 2 |
| 7-15 | H | 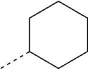 | 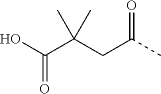 | Ex 1 |
| 7-16 | H | 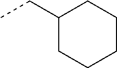 | 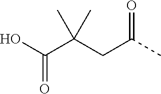 | Ex 2 |
| 7-17 | H | 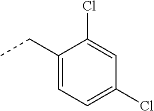 | 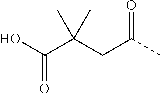 | Ex 2 |
| 7-18 | H | 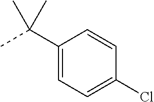 | 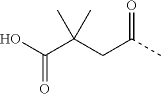 | Ex 2 |
| 7-19 | 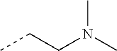 | 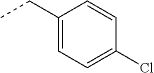 | 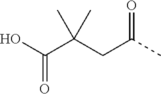 | Ex 4 |
| 7-20 | H | 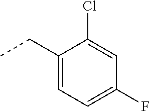 | 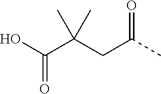 | Ex 2 |
| 7-21 | H | 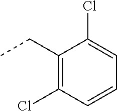 | 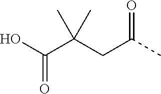 | Ex 2 |
| 7-22 | H | 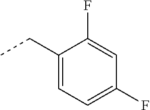 | 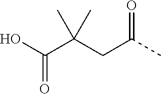 | Ex 2 |
| 7-23 | H | 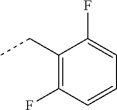 | 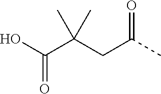 | Ex 2 |
| 7-24 | H | 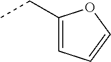 | 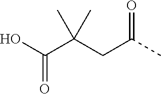 | Ex 2 |

### Example 8

### 4-{[(3aR,5aR,5bR,7aR,9S,11aR,11bR,13aS)-3a-({[(4-Chlorophenyl)methyl]amino}acetyl)-5a,5b,8,8,11a-pentamethyl-1-(1-methylethyl)-2-oxo-3,3a,4,5,5a,5b,6,7,7a,8,9,10,11,11a,11b,12,13,13a-octadecahydro-2H-cyclopenta[a]chrysen-9-yl]oxy}-2,2-dimethyl-4-oxobutanoic acid

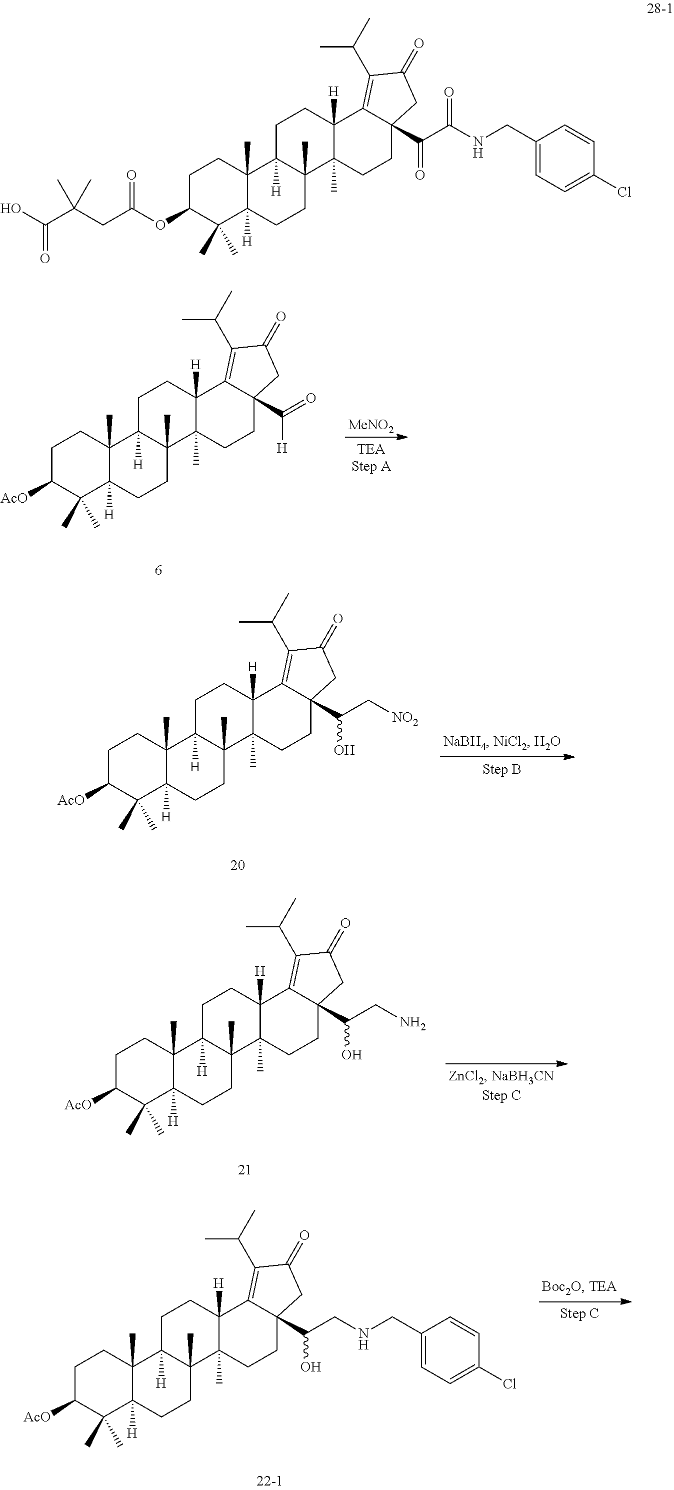

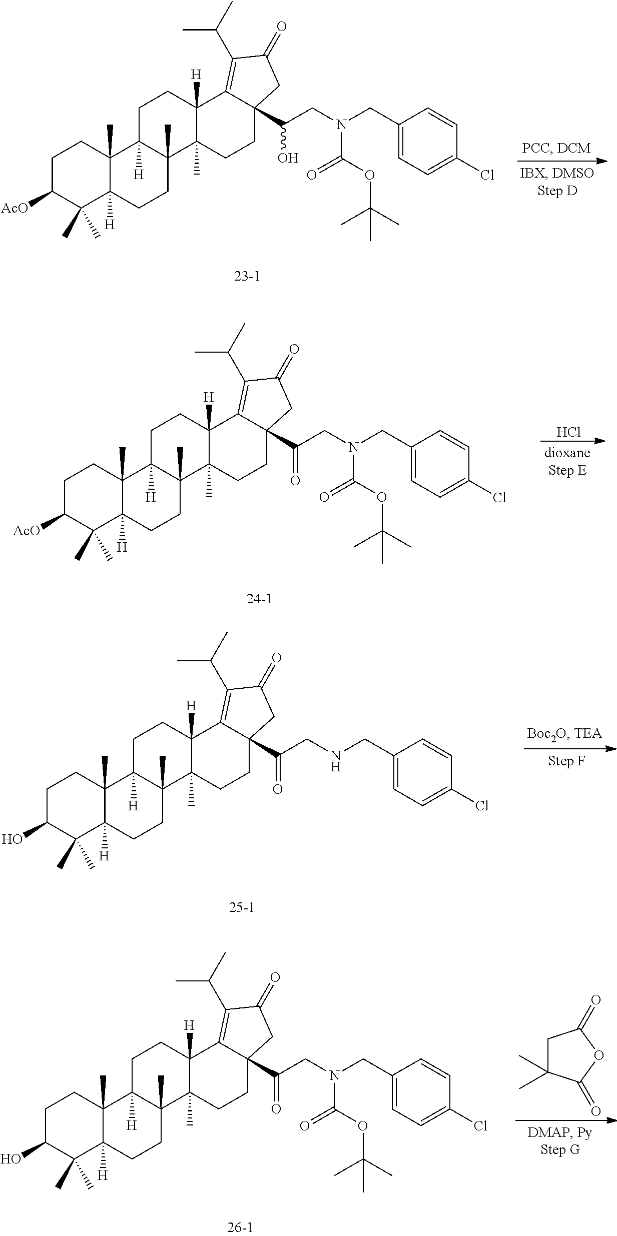

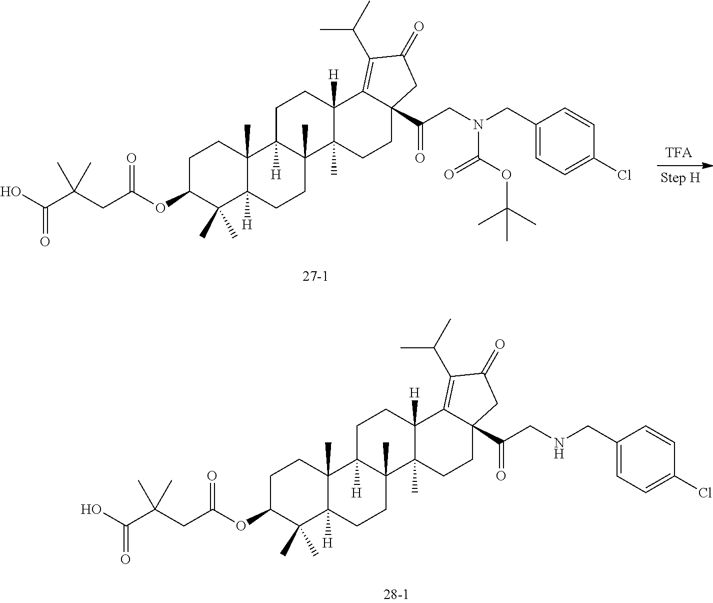

### Step A: Intermediate 20

To a mixture of the intermediate 6 (300 mg, 0.604 mmol) and MeNO₂ (7.5 mL, 139 mmol) was added Et₃N (0.6 mL, 4.30 mmol) at room temperature. After stirring overnight, the reaction was quenched with water (1.0 mL), and partitioned between EtOAc (50 mL) and water (25 mL). The organic phase was washed with saturated brine (50 mL), dried over sodium sulfate and evaporated to dryness in vacuo. The obtained residue was purified by column chromatography on silica gel (Hex:EtOAc=4:1) to afford the intermediate 20 (335 mg, 99%) as a white solid.

### Step B: Intermediate 21

To a solution of NiCl₂.6H₂O (0.929 g, 7.17 mmol) in MeOH (125 mL) was added portionwise sodium borohydride (0.271 g, 7.17 mmol) at 0° C. After stirring for 30 min, the intermediate 20 (2 g, 3.59 mmol) was added, followed by the addition of another portion of sodium borohydride (2.44 g, 64.53 mmol). The reaction mixture was stirred at 0° C. for another 30 min, and filtration was performed to remove the insoluble material. The filtrate was concentrated to dryness in vacuo. The obtained residue was dissolved into EtOAc (100 mL), and the organic phase was washed with water (40 mL), brine (40 mL), dried over sodium sulfate, and evaporated to dryness in vacuo to give the intermediate 21 (1.79 g, 95%) as an off-white solid. LC/MS: m/z calculated 527.3, found 528.2 (M+1)+.

### Step C: Intermediates 22-1 and 23-1

To a suspension of the intermediate 21 (450 mg, 0.853 mmol) and 4-chlorobenzaldehyde (120 mg, 0.853 mmol) in MeOH (30 mL) was added ZnCl₂ (69.7 mg, 0.083 mmol) at room temperature. After stirring at room temperature for 2 h, sodium cyanoborohydride (107 mg, 0.25 mmol) was added, and the resulting mixture was stirred for another 2 h to give the intermediate 22-1.

To the reaction mixture obtained above were added Boc₂O (0.297 mL, 1.279 mmol) and Et₃N (0.238 mL, 1.705 mmol). After stirring at room temperature overnight, the insoluble material was removed by filtration, and the filtrate was concentrated to dryness under reduced pressure. The obtained residue was dissolved into EtOAc (50 mL), and the organic phase was washed with water (25 mL), brine (25 m), dried over magnesium sulfate, and evaporated to dryness in vacuo to give a residue, which was purified by Prep-TLC to afford the intermediate 23-1 (164 mg, 25.6%) as a white solid.

### Step D: Intermediate 24-1

To a solution of the intermediate 23-1 (130 mg, 0.173 mmol) in DCM (5 mL) were added PCC (372 mg, 1.728 mmol) and silica gel (120 mg) at room temperature. After stirring at room temperature for 8 h, the insoluble material was removed by filtration and the filtrate was concentrated to dryness under reduced pressure. The obtained residue was purified by column chromatography on silica gel (Hex:EtOAc=1:6) to give the intermediate 24-1 (92 mg, 71%) as a white solid. LC/MS: m/z calculated 785.4, found 686.0 (M−Boc+1)+.

### Step E: Intermediate 25-1

To a solution of the intermediate 24-1 (28 mg, 0.037 mmol) in 1,4-dioxane (2.5 mL) was added conc. HCl (1.0 mL, 32.9 mmol). After stirring at room temperature overnight, the reaction mixture was diluted with EtOAc (50 mL), neutralized with saturated sodium bicarbonate solution, and partitioned between EtOAc (50 mL) and saturated sodium bicarbonate (25 mL). The organic phase was washed with saturated sodium bicarbonate (25 mL), brine (25 mL), dried over sodium sulfate and evaporated to dryness in vacuo to give the intermediate 25-1 (22.3 mg, 98%) as a colorless oil. LC/MS: m/z calculated 607.3, found 608.1 (M+1)+.

### Step F: Intermediate 26-1

To a solution of the intermediate 25-1 (79 mg, 0.13 mmol) and Boc₂O (28.3 mg, 0.13 mmol) in DCM (5 mL) was added Et₃N (18 μL, 0.13 mmol). After stirring at room temperature for 1 h, the reaction was quenched with water (1.0 mL), and partitioned between EtOAc (25 mL) and water (10 mL). The organic phase was washed with saturated sodium bicarbonate (10 mL), brine (10 mL), dried over sodium sulfate and evaporated to dryness in vacuo to give a residue, which was purified by column chromatography on silica gel (Hex:EtOAc=5:1) to give the intermediate 26-1 (57 mg, 62%) as a white foam.

### Step G: Intermediate 27-1

To a solution of the intermediate 26-1 (26 mg, 0.037 mmol) in anhydrous pyridine (2 mL) were added DMAP (22.42 mg, 0.184 mmol) and 3,3-dimethyldihydro-2,5-furandione (47 mg, 0.367 mmol). After it was stirred at 80° C. overnight, the reaction mixture was diluted with EtOAc (30 mL). The organic phase was washed with aqueous HCl (2 N, 10 mL), brine (20 mL), dried over sodium sulfate and evaporated to dryness in vacuo to give a residue, which was purified by column chromatography on silica gel (Hex:EtOAc 4:1) to afford the intermediate 27-1 (15 mg, 48.9%) as a white foam. LC/MS: m/z calculated 835.4, found 858.4 (M+Na)⁺.

### Step H: Compound 28-1

To a solution of the intermediate 27-1 (27 mg, 0.032 mmol) in DCM (1.0 mL) was added TFA (0.5 mL, 6.49 mmol) dropwise. After stirring at room temperature for 1 h, the reaction mixture was neutralized by aqueous sodium bicarbonate (25 mL). The reaction mixture was partitioned between EtOAc (50 mL) and aqueous sodium bicarbonate (25 mL) and the organic phase was washed with brine (25 mL), dried over sodium sulfate and evaporated to dryness in vacuo to afford a residue, which was purified by Prep-HPLC to give the title compound 28-1 (9 mg, 29.5%). LC/MS: m/z calculated 735.3, found 736.3 (M+1)+.

### Example 9

### 4-{[(3aR,5aR,5bR,7aR,9S,11aR,11bR,13aS)-3a-{[(2-Furanylmethyl)amino]acetyl}-5a,5b,8,8,11a-pentamethyl-1-(1-methylethyl)-2-oxo-3,3a,4,5,5a,5b,6,7,7a,8,9,10,11,11a,11b,12,13,13a-octadecahydro-2H-cyclopenta[a]chrysen-9-yl]oxy}-2,2-dimethyl-4-oxobutanoic acid

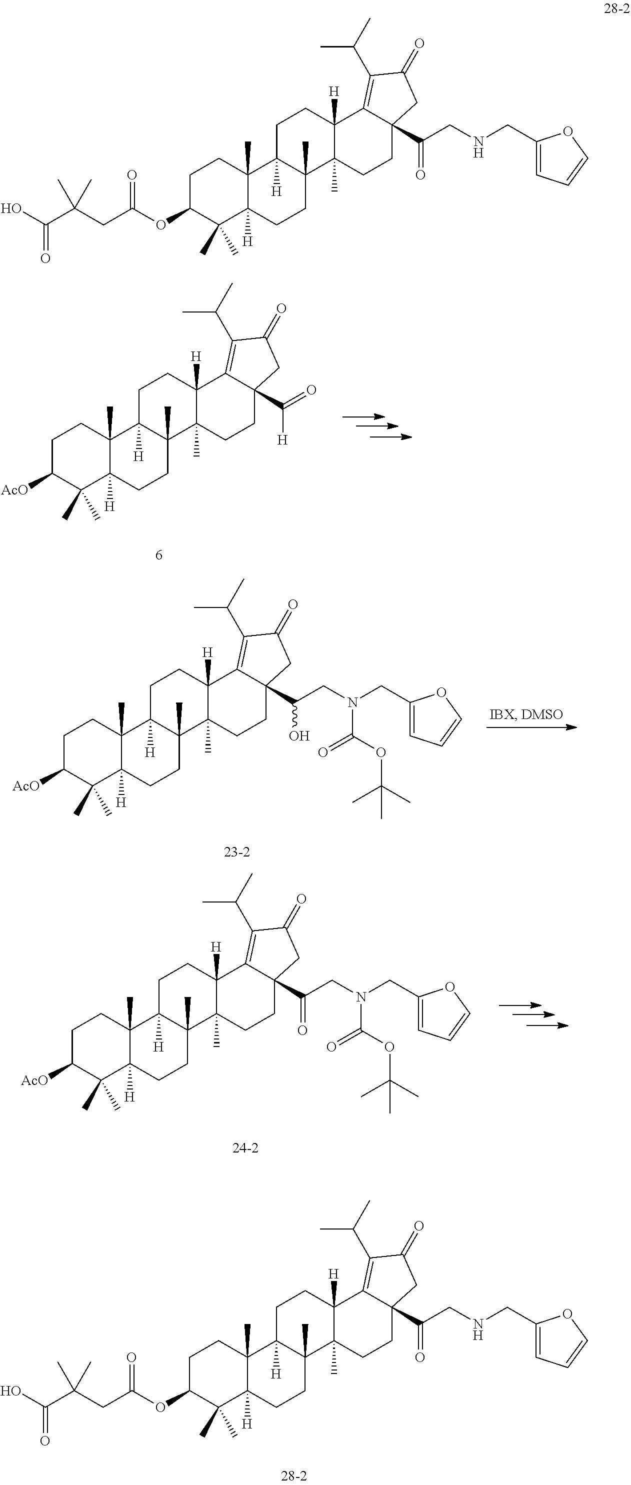

### Intermediate 24-2

To a solution of the intermediate 23-2 (1.46 g, 1.24 mmol) in DMSO (10 mL), which was prepared according to the description in Example 8, was added IBX (2 g, 7.14 mmol). After it was stirred at 60° C. for 2 h, the reaction mixture was diluted with EtOAc (50 mL). The organic phase was washed with water (30 mL), brine (30 mL), dried over sodium sulfate and evaporated to dryness in vacuo to afford a residue, which was purified by column chromatography on silica gel (PE:EtOAc=100:0 to 85:15) to give the intermediate 24-2 (180 mg, 20.6%) as a white solid. LC/MS: m/z calculated 727.3, found 728.3 (M+1)+.

### Compound 28-2

Compound 28-2 was prepared as a white solid from the intermediate 24-2 by a similar procedure used in Example 8. ¹HNMR (400 MHz, CD₃OD) δ ppm 7.54 (1H, d, J=1.2 Hz), 6.52 (1H, d, J=3.2 Hz), 6.41 (1H, dd, J=3.2, 2.0 Hz), 4.39 (1H, dd, J=10.8, 5.6 Hz), 4.21 (2H, s), 3.99 (1H, d, J=18.0 Hz), 3.89 (1H, d, J=18.0 Hz), 3.27-3.19 (1H, m), 2.54 (1H, d, J=16.0 Hz), 2.46 (1H, d, J=16.0 Hz), 2.49-2.39 (2H, m), 2.34 (1H, d, J=19.2 Hz), 2.13 (1H, d, J=19.2 Hz), 1.99-1.81 (2H, m), 1.73-1.18 (13H, m), 1.17 (3H, s), 1.16 (3H, s), 1.13 (3H, s), 1.11 (3H, s), 1.04-0.92 (3H, m), 0.96 (3H, s), 0.90 (3H, s), 0.85 (3H, s), 0.77 (6H, s). LC/MS: m/z calculated 691.3, found 692.1 (M+1)+.

### Example 10

### 2,2-Dimethyl-4-oxo-4-[((3aR,5aR,5bR,7aR,9S,11aR,11bR,13aS)-5a,5b,8,8,11a-pentamethyl-1-(1-methylethyl)-2-oxo-3a-{[(3-pyridinylmethyl)amino]acetyl}-3,3a,4,5,5a,5b,6,7,7a,8,9,10,11,11a,11b,12,13,13a-octadecahydro-2H-cyclopenta[a]chrysen-9-yl)oxy]butanoic acid

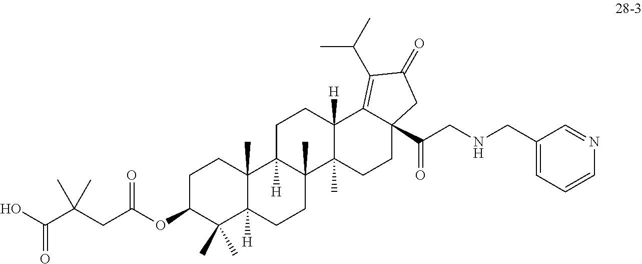

A similar procedure as described in Example 8 and 9 was used to prepare the title compound as a light yellow solid. ¹HNMR (400 MHz, CDCl₃) δ ppm 8.51-8.48 (2H, m), 7.68 (1H, d, J=8.0H), 7.22 (1H, br), 4.49 (1H, dd, J=11.2, 4.8 Hz), 3.71 (2H, s), 3.41 (1H, d, J=18.4 Hz), 3.30 (1H, d, J=18.4 Hz), 3.17-3.11 (1H, m), 2.68-0.74 (52H, m). LC/MS: m/z calculated 702.4, found 703.3 (M+1)+.

### Example 11

### 2-2,Dimethyl-4-oxo-4-({(3aR,5aR,5bR,7aR,9S,11aR,11bR,13aS)-5a,5b,8,8,11a-pentamethyl-1-(1-methylethyl)-2-oxo-3a-[({[3-(trifluoromethyl)phenyl]methyl}amino)acetyl]-3,3a,4,5,5a,5b,6,7,7a,8,9,10,11,11a,11b,12,13,13a-octadecahydro-2H-cyclopenta[a]chrysen-9-yl}oxy)butanoic acid

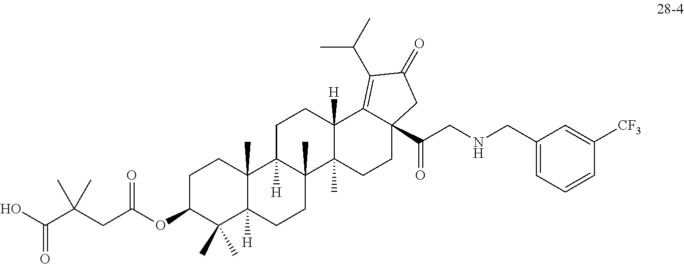

A similar procedure as described in Example 8 and 9 was used to prepare the title compound. ¹HNMR (400 MHz, CD₃OD) δ ppm 7.86-7.66 (4H, m), 4.47 (1H, dd, J=11.2, 5.2 Hz), 4.31 (2H, s), 4.22 (1H, d, J=17.6 Hz), 4.11 (1H, d, J=17.6 Hz), 3.38-3.27 (1H, m), 2.64-0.87 (52H, m). LC/MS: m/z calculated 769.4, found 770.3 (M+1)+.

### Example 12

### 4-((3aR,5aR,5bR,7aR,9S,11aR,11bR,13aS)-1-isopstickyl-5a,5b,8,8,11a-pentamethyl-3a-(2-(4-methylpiperazin-1-yl)acetyl)-2-oxo-3,3a,4,5,5a,5b,6,7,7a,8,9,10,11,11a,11b,12,13,13a-octadecahydro-2H-cyclopenta[a]chrysen-9-yloxy)-2,2-dimethyl-4-oxobutanoic acid

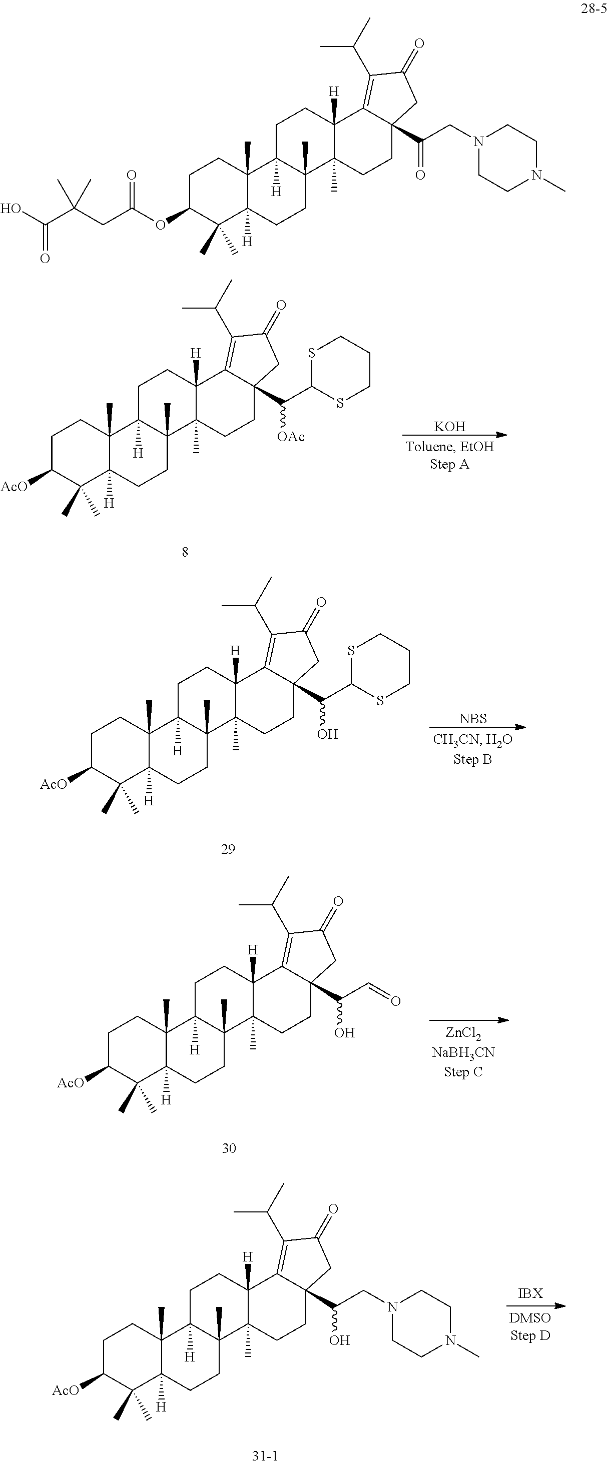

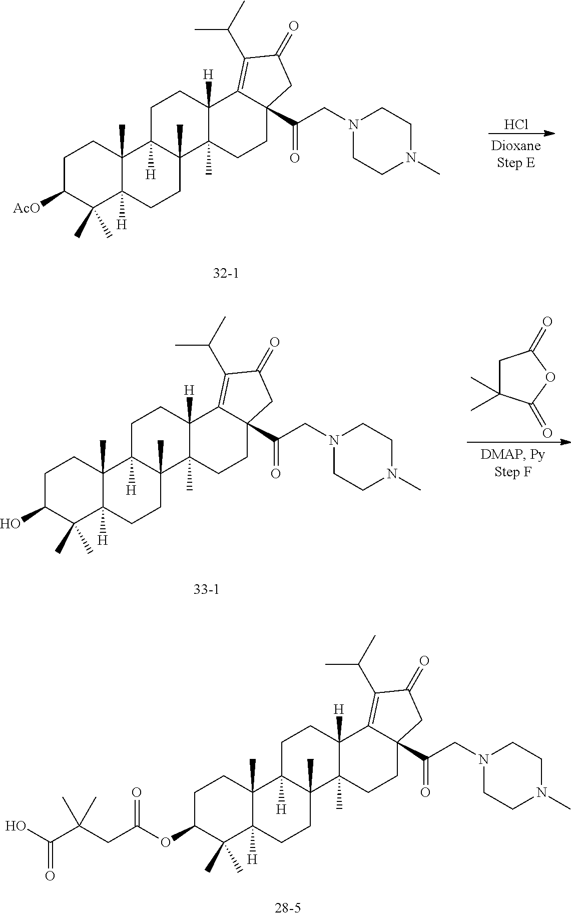

### Step A: Intermediate 29

A suspension of the intermediate 8 (863 mg, 1.31 mmol) and KOH (367 mg, 6.55 mmol) in a mixture of 1:1 of toluene (20 mL) and EtOH (20 mL) was stirred vigorously at room temperature for 1 h. After it was neutralized with aqueous HCl (1N), the reaction mixture was evaporated to dryness. The obtained residue was taken up in DCM (200 mL) and the organic phase was washed with water (200 mL), saturated sodium bicarbonate solution (100 mL×2), dried over sodium sulfate and evaporated to dryness in vacuo to give a residue, which was purified by column chromatography on silica gel (EtOAc:PE=1:30 to 1:15) to afford the intermediate 29 (464 mg, 57.4%) as a white solid. LC/MS: m/z calculated 616.3, found 617.0 (M+1)+.

### Step B: Intermediate 30

To a solution of NBS (803 mg, 4.51 mmol) in acetonitrile (20 mL) and water (5 mL) was added the intermediate 29 (464 mg, 0.752 mmol) at room temperature. After stirring at room temperature for 0.5 h, the reaction was quenched with aqueous sodium sulfite, and concentrated. The obtained residue was dissolved into ether (30 mL), and the organic phase was washed with brine (15 mL), dried over sodium sulfate and evaporated to dryness to give the intermediate 30 (541 mg, 96%) as a white solid. LC/MS: m/z calculated 526.3, found 527.1 (M+1)+.

### Step C: Intermediate 31-1

A suspension of the intermediate 30 (450 mg, 0.598 mmol), 1-methylpiperazine (0.133 mL, 1.196 mmol), and zinc chloride (48.9 mg, 0.359 mmol) in MeOH (20 mL) was heated at 70° C. for 0.5 h. After cooling down to room temperature, sodium cyanoborohydride (225 mg, 3.59 mmol) was added in one portion. The reaction mixture was stirred at room temperature for another 17 h before it was diluted with EtOAc (50 mL). The organic phase was washed with saturated aqueous sodium bicarbonate solution (30 mL), dried over sodium sulfate and evaporated to dryness in vacuo to give a residue, which was purified by Prep-HPLC to afford the intermediate 31-1 (291 mg, 80%) as a white solid. LC/MS: m/z calculated 610.3, found 611.4 (M+1)+.

### Step D: Intermediate 32-1

A solution of the intermediate 31-1 (50 mg, 0.082 mmol), and IBX (68.8 mg, 0.246 mmol) in DMSO (5 mL) was stirred at 60° C. for 4 h. After that, the reaction mixture was diluted with EtOAc (30 mL), and the organic phase was washed with saturated sodium bicarbonate (15 mL), water (15 mL), brine (15 mL), dried over sodium sulfate and evaporated to dryness in vacuo to give the intermediate 32-1 (42 mg, 84%) as a white foam. LC/MS: m/z calculated 608.3, found 609.3 (M+1)+.

### Step E: Intermediate 33-1

A solution of the intermediate 32-1 (42 mg, 0.069 mmol) in 1,4-dioxane (4 mL) and conc. HCl (2 mL, 65.8 mmol) was stirred at room temperature overnight. After that, the reaction mixture was evaporated to dryness. The obtained residue was dissolved into EtOAc (30 mL), and the organic phase was washed with saturated sodium bicarbonate (15 mL), brine (15 mL), dried over sodium sulfate and evaporated in vacuo to give the intermediate 33-1 (42 mg, 97%) as a white foam. LC/MS: m/z calculated 566.3, found 567.4 (M+1)+.

### Step F: Compound 28-5

A solution of the intermediate 33-1 (80 mg, 0.141 mmol), 3,3-dimethyl-dihydrofuran-2,5-dione (362 mg, 2.82 mmol) and DMAP (86 mg, 0.706 mmol) in pyridine (0.8 mL) was stirred under an atmosphere of Nitrogen at 100° C. for 3 h. After cooling down to room temperature, the reaction mixture was diluted with EtOAc (30 mL). The organic phase was washed with brine, dried over sodium sulfate and evaporated to dryness. The obtained residue was purified by Prep-HPLC to give the compound 28-5 (50 mg, 38%) as a white foam. ¹HNMR (400 MHz, CD₃OD) δ ppm 4.50 (1H, dd, J=10.8, 5.6 Hz), 3.62-3.40 (2H, m), 3.38-3.20 (4H, m), 2.99-2.81 (6H, m), 2.68-2.53 (4H, m), 2.40 (1H, d, J=19.2 Hz), 2.15 (1H, d, J=19.2 Hz), 2.10-1.93 (2H, m), 1.83-0.87 (44H, m). LC/MS: m/z calculated 694.3, found 695.4 (M+1)+.

The compounds in Table 2 were prepared in a manner similar to the indicated procedures above. In each case, X is carbonyl and Y is CH₂.

TABLE 2

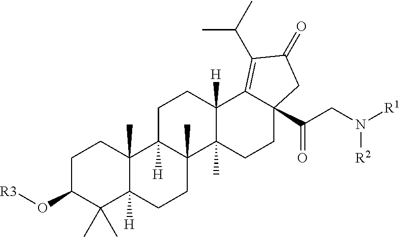

| Example | | | | Synthetic |
|---|---|---|---|---|
| No. | R¹ | R² | R³ | Procedure |
| 12-1 | H | 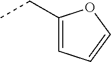 | 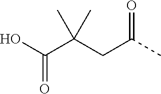 | Ex 9 |
| 12-2 | 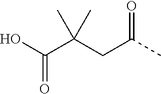 | 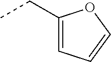 | 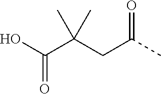 | Ex 9 |
| 12-3 | 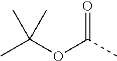 | 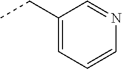 | 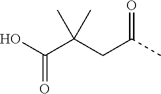 | Ex 9 |
| 12-4 | H | 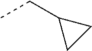 | 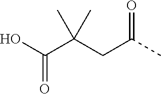 | Ex 9 |
| 12-5 | H | 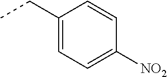 | 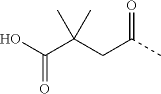 | Ex 9 |
| 12-6 | H | 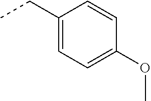 | 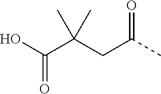 | Ex 9 |
| 12-7 | H | 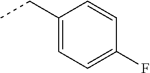 | 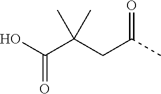 | Ex 9 |
| 12-8 | H | 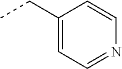 | 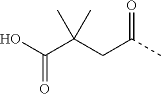 | Ex 9 |
| 12-9 | H | 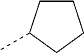 | 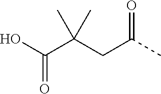 | Ex 9 |
| 12-10 | H | 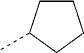 | 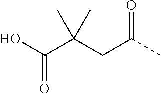 | Ex 9 |
| 12-11 | H | 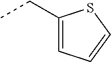 | 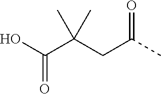 | Ex 9 |
| 12-12 | H | H | 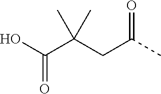 | Ex 8 |
| 12-13 | H | 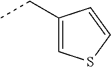 | 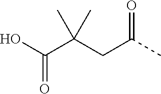 | Ex 8 |
| 12-14 | H | 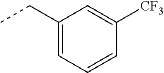 | 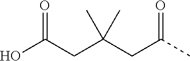 | Ex 8 |
| 12-15 | H | 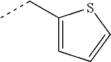 | 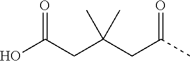 | Ex 9 |
| 12-16 | H | 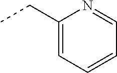 | 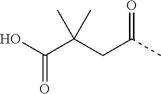 | Ex 9 |
| 12-17 | CH₃ | 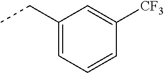 | 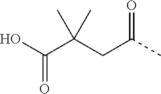 | Ex 9 |
| 12-18 | H | 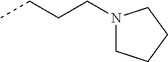 | 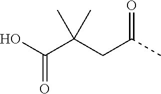 | Ex 12 |
| 12-19 | CH₃ | 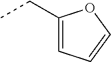 | 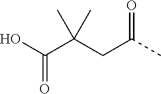 | Ex 8 |
| 12-20 | H | 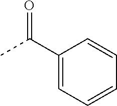 | 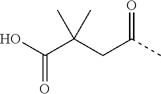 | Ex 9 |
| 12-21 | CH₃ | 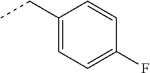 | 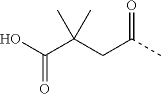 | Ex 9 |
| 12-22 | CH₃ | 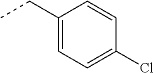 | 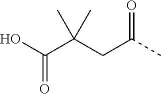 | Ex 8 |
| 12-23 | H | 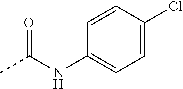 | 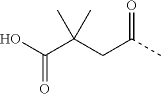 | Ex 9 |
| 12-24 | CH₃ | 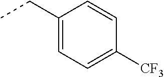 | 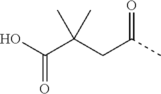 | Ex 12 |
| 12-25 | CH₃ | 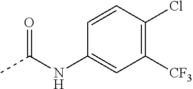 | 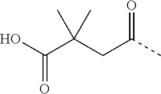 | Ex 9 |
| 12-26 | H | 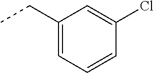 | 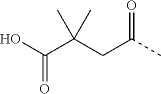 | Ex 8 |
| 12-27 | H | 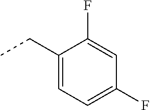 | 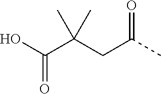 | Ex 9 |
| 12-28 | CH₃ | 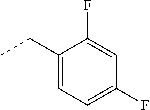 | 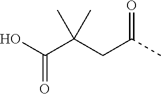 | Ex 9 |
| 12-29 | H | 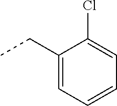 | 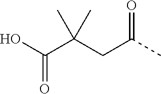 | Ex 9 |
| 12-30 | CH₃ | 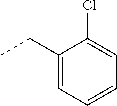 | 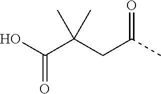 | Ex 9 |
| 12-31 | 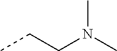 | 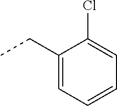 | 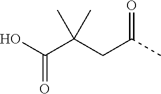 | Ex 9 |
| 12-32 | CH₃ | 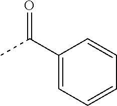 | 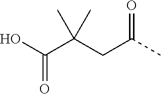 | Ex 9 |
| 12-33 | CH₃ | 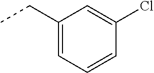 | 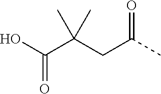 | Ex 9 |
| 12-34 | 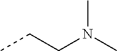 | 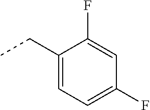 | 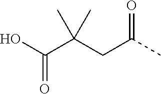 | Ex 9 |
| 12-35 | H | 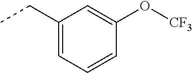 | 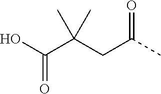 | Ex 8 |
| 12-36 | CH₃ | 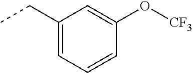 | 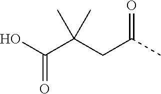 | Ex 8 |
| 12-37 | 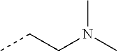 | 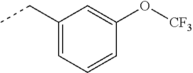 | 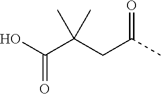 | Ex 8 |
| 12-38 | 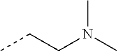 | 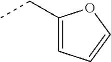 | 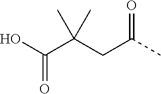 | Ex 8 |

### Example 13

### 4-((3aS,5aR,5bR,7aR,9S,11aR,11bR,13aS)-3a-(2-(4-Chlorobenzylamino)acetyl)-1-isopstickyl-5a,5b,8,8,11a-pentamethyl-3,3a,4,5,5a,5b,6,7,7a,8,9,10,11,11a,11b,12,13,13a-octadecahydro-2H-cyclopenta[a]chrysen-9-yloxy)-2,2-dimethyl-4-oxobutanoic acid

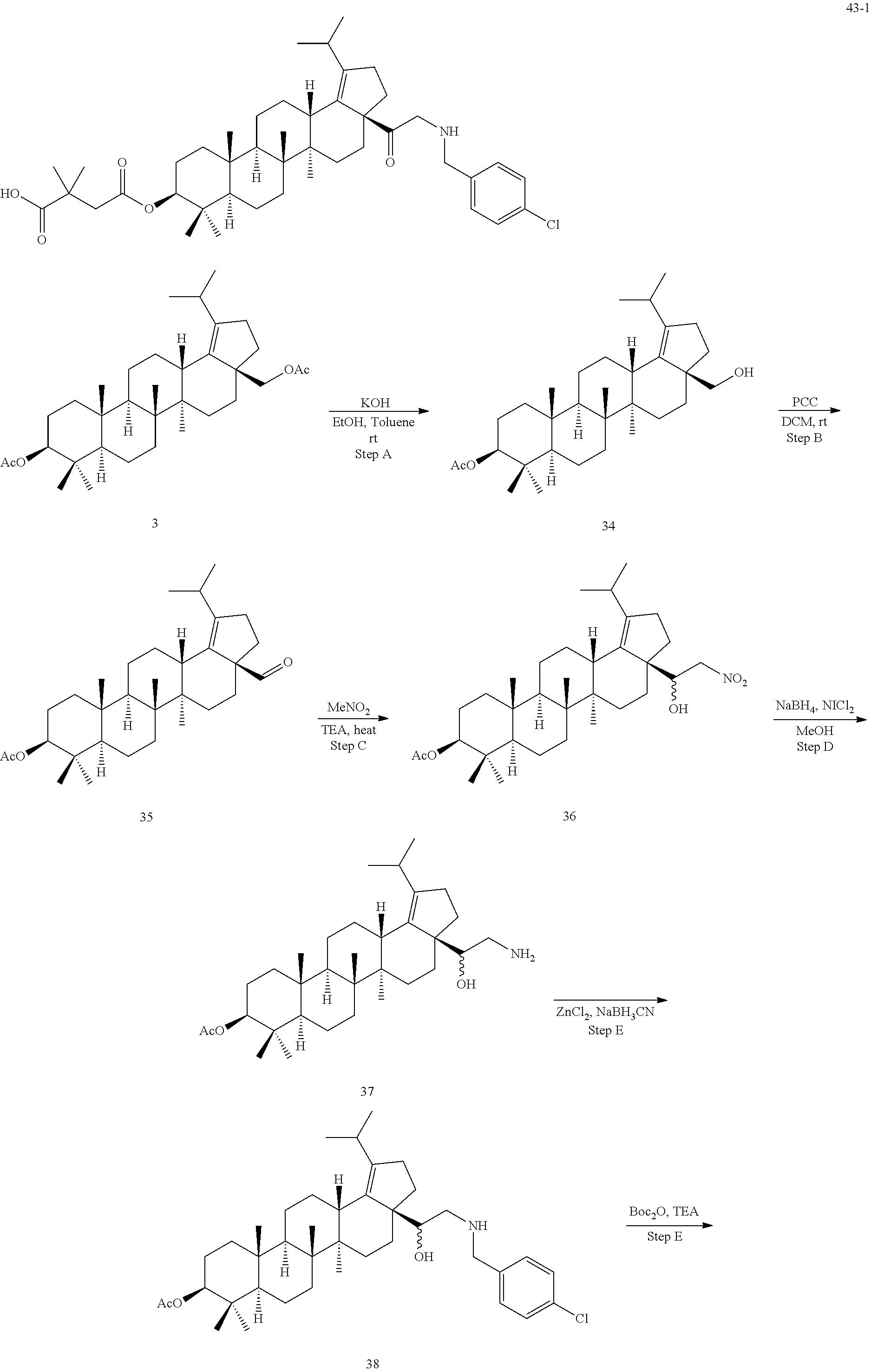

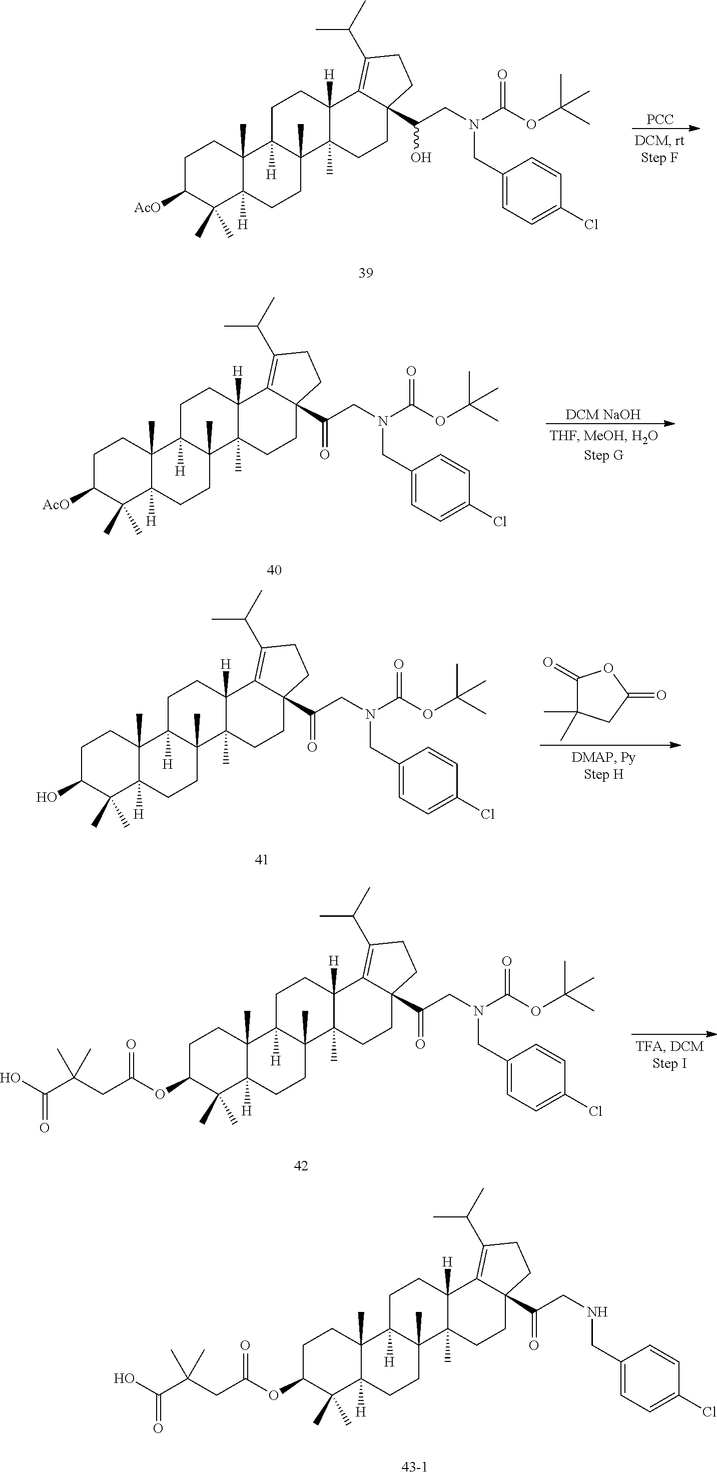

### Step A: Intermediate 34

To a solution of the intermediate 3 (5 g, 7.59 mmol) in EtOH (100 mL) and toluene (100 mL) was added KOH (0.51 g, 9.11 mmol). After stirring at room temperature for 4 h, the reaction mixture was then partitioned between water (500 mL) and EtOAc (500 mL). The organic phase was washed with water (200 mL×3), brine (100 mL), and dried over sodium sulfate. Removal of the solvent gave a residue, which was purified by column chromatography on silica gel (Hex:EtOAc=6:1 to 4:1) to afford the intermediate 34 (2.5 g, 67.9%) as a white solid. ¹H NMR (400 Hz, CDCl₃) δ ppm 4.50-4.67 (1H, m), 3.68 (1H, d, J=10.4 Hz), 3.32 (1H, d, J=10.4 Hz), 3.23-3.15 (1H, m), 2.42-2.28 (3H, m), 2.05 (3H, s), 2.02-1.89 (2H, m), 1.77-0.83 (40H, m).

### Step B: Intermediate 35

To a solution of the intermediate 34 (3 g, 6.19 mmol) in DCM (75 mL) at room temperature were added PCC (4 g, 18.57 mmol) and silica gel (3.0 g). After stirring at room temperature for 2 h, the reaction was quenched with water (100 mL). The organic phase was washed with saturated sodium bicarbonate (50 mL), dried over sodium sulfate and concentrated in vacuo to give a residue, which was purified by column chromatography on silica gel (Hex:EtOAc=10:1) to afford the intermediate 35 (3 g, 100%) as a white solid. ¹H NMR (400 Hz, CDCl₃) δ ppm 9.43 (1H, s), 4.50-4.46 (1H, m), 3.25-3.21 (1H, m), 2.43-2.02 (5H, m), 2.04 (3H, m), 2.00-1.93 (1H, m), 1.75-0.81 (38H, m).

### Step C: Intermediate 36

To a solution of the intermediate 36 (5 g, 10.36 mmol) in MeNO₂ (128 mL, 2382 mmol) was added Et₃N (10.11 mL, 72.5 mmol). After it was stirred at 60° C. overnight, the reaction mixture was partitioned between water and EtOAc (100 mL each). The organic phase was washed with water (20 mL×3), brine (20 mL), and dried over sodium sulfate. Removal of the solvent gave a residue, which was purified by column chromatography on silica gel (Hexane:EtOAc=10:1 to 6:1) to afford the intermediate 36 (2.8 g, 49.7%) as a white powder. LC/MS: m/z calculated 543.4, found 566.3 (M+Na⁺)+.

### Step D: Intermediate 37

To a solution of the intermediate 36 (2.8 g, 5.15 mmol) in MeOH (166 mL) were added nickel(II) chloride (1.67 g, 12.87 mmol) and sodium borohydride (4.87 g, 129 mmol) at 0° C. After stirring at 0° C. for 10 min, the reaction mixture was partitioned between water and EtOAc (200 mL each), and the organic phase was washed with water (100 mL×3), brine (50 mL), and dried over sodium sulfate. Removal of the solvent gave the intermediate 37 (2.65 g, 100%) as a solid. LC/MS: m/z calculated 513.4, found 514.3 (M+1)+.

### Step E: Intermediate 39

To a solution of the intermediate 37 (350 mg, 0.613 mmol) and 4-chlorobenzaldehyde (86 mg, 0.613 mmol) in MeOH (15 mL) and dichloroethane (DCE, 15 mL) was added zinc chloride (50.1 mg, 0.368 mmol). After the reaction mixture was stirred at 80° C. for 1 h and cooled down to room temperature, sodium cyanoborohydride (57.8 mg, 0.92 mmol) was added. The resulting mixture was stirred at room temperature for another 1 h to give the intermediate 38.

To the reaction mixture obtained above were added Et₃N (0.18 mL, 1.38 mmol) and di-tert-butyl dicarbonate (0.157 mL, 0.674 mmol). After stirring at room temperature for 30 min, the reaction mixture was partitioned between water (20 mL) and EtOAc (100 mL). The organic phase was washed with water (30 mL×3), brine (20 mL), and dried over sodium sulfate. Removal of the solvent gave a residue, which was purified by column chromatography on silica gel (Hex:EtOAc=15:1) to afford the intermediate 39 (125 mg, 27.6%) as a white solid.

### Step F: Intermediate 40

To a solution of the intermediate 39 (120 mg, 0.162 mmol) in DCM (10 mL) were added PCC (35 mg, 0.162 mmol) and silica gel (100 mg). After stirring at room temperature for 2 h, the insoluble material was removed by filtration and the filtrate was concentrated to afford the intermediate 40 (110 mg, 92%) as a white solid.

### Step G: Intermediate 41

To a solution of NaOH (597 mg, 14.94 mmol) in MeOH (1 mL), THF (1 mL), and water (0.5 mL) was added the intermediate 40 (110 mg, 0.149 mmol). After stirring at room temperature for 1 h, the reaction was diluted with water (20 mL), and extracted with EtOAc (50 mL). The organic phase was washed with brine (20 mL), dried over sodium sulfate, and evaporated in vacuo to give the intermediate 41 (100 mg, 96%) as a white solid.

### Step H: Intermediate 42

To a solution of the intermediate 41 (100 mg, 0.144 mmol) in anhydrous pyridine (2 mL) were added DMAP (106 mg, 0.864 mmol) and 3,3-dimethyldihydro-2,5-furandione (369 mg, 2.88 mmol). After the reaction mixture was heated at 80° C. overnight, the solvent was removed in vacuo and the residue was taken up in DCM (50 mL). The organic phase was washed with aqueous HCl (0.5 N, 20 mL), water (2×30 mL), brine (30 mL), dried over sodium sulfate, and evaporated in vacuo to give a residue, which was purified by column chromatography on silica gel (Hex:EtOAc=15:1) to give the intermediate 42 (56 mg, 44.9%) as a white foam.

### Step I: Compound 43-1

To a solution of the intermediate 42 (45 mg, 0.055 mmol) in DCM (1 mL) was added TFA (0.5 mL, 6.49 mmol). After stirring at room temperature for 1 h, removal of the solvent gave a residue, which was purified by Prep-HPLC to afford the compound 43-1 (38 mg, 77%) as a white solid. ¹H NMR (400 Hz, CD₃OD) δ ppm 7.42-7.36 (4H, m), 4.40-4.36 (1H, m), 4.15 (2H, dd, J=16.8, 13.2 Hz), 3.93 (1H, d, J=17.6 Hz), 3.85 (1H, d, J=17.6 Hz), 3.19-3.14 (m, 1H), 2.57-1.98 (5H, m), 1.97-0.77 (48H, m). LC/MS: m/z Calculated 721.4, found 722.1 (M+1)+.

### Example 14

### 4-((3aS,5aR,5bR,7aR,9S,11aR,11bR,13aS)-3a-(2-(4-fluorobenzylamino)acetyl)-1-isopstickyl-5a,5b,8,8,11a-pentamethyl-3,3a,4,5,5a,5b,6,7,7a,8,9,10,11,11a,11b,12,13,13a-octadecahydro-2H-cyclopenta[a]chrysen-9-yloxy)-2,2-dimethyl-4-oxobutanoic acid

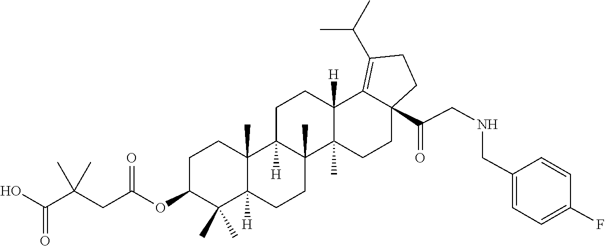

A similar procedure as described in Example 13 was used to prepare the title compound as a white solid. ¹H NMR (400 Hz, MeOD) δ ppm 7.55-7.53 (2H, m), 7.24-7.20 (2H, m), 4.50-4.40 (1H, m), 4.27-4.24 (2H, dd, J=11.6, 11.2 Hz), 4.03-3.98 (2H, dd, J=18.8, 14.4 Hz), 3.31-3.25 (1H, m), 2.67-2.46 (4H, m), 2.34-2.31 (1H, m), 2.12-0.88 (48H, m). LC/MS: m/z Calculated 705.4, found 706.4 (M+1)+.

### Example 15

### 4-((3aS,5aR,5bR,7aR,9S,11aR,11bR,13aS)-3a-(2-(Furan-2-ylmethylamino)acetyl)-1-isopstickyl-5a,5b,8,8,11a-pentamethyl-3,3a,4,5,5a,5b,6,7,7a,8,9,10,11,11a,11b,12,13,13a-octadecahydro-2H-cyclopenta[a]chrysen-9-yloxy)-2,2-dimethyl-4-oxobutanoic acid

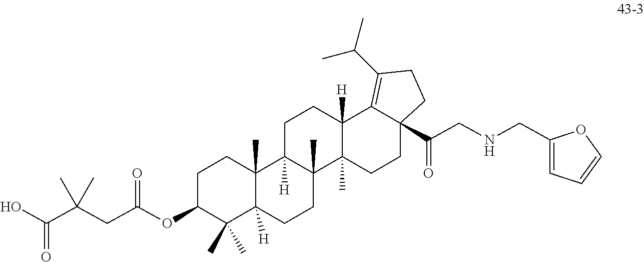

A similar procedure as described in Example 13 was used to prepare the title compound as a white solid. ¹H NMR (400 Hz, MeOD) δ ppm 7.55 (1H, br), 6.54 (1H, d, J=3.2 Hz), 6.43-6.42 (1H, m), 4.40-4.36 (1H, m), 4.23 (2H, s), 3.87 (2H, s), 3.19-3.16 (1H, m), 2.57-2.22 (5H, m), 2.05-0.77 (48H, m). LC/MS: m/z Calculated 677.4, found 678.4 (M+1)+.

### Example 16

### 4-((3aS,5aR,5bR,7aR,9S,11aR,11bR,13aS)-3a-(2-(cyclopentylamino)acetyl)-1-isopstickyl-5a,5b,8,8,11a-pentamethyl-3,3a,4,5,5a,5b,6,7,7a,8,9,10,11,11a,11b,12,13,13a-octadecahydro-2H-cyclopenta[a]chrysen-9-yloxy)-2,2-dimethyl-4-oxobutanoic acid

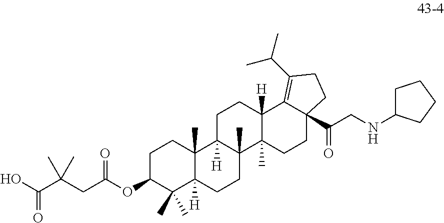

A similar procedure as described in Example 13 was used to prepare the title compound as a white solid. ¹H NMR (400 Hz, MeOD) δ ppm 4.41-4.37 (1H, m), 3.88 (2H, br), 3.47 (1H, br), 3.22-3.17 (1H, m), 2.57-2.45 (4H, m), 2.22 (1H, d, J=13.2 Hz), 2.09 (1H, d, J=13.6 Hz), 1.99-0.77 (55H, m). LC/MS: m/z Calculated 665.5, found 666.4 (M+1)+.

### Example 17

### 4-((3aS,5aR,5bR,7aR,9S,11aR,11bR,13aS)-1-Isopstickyl-5a,5b,8,8,11a-pentamethyl-3a-(2-(4-methylpiperazin-1-yl)acetyl)-3,3a,4,5,5a,5b,6,7,7a,8,9,10,11,11a,11b,12,13,13a-octadecahydro-2H-cyclopenta[a]chrysen-9-yloxy)-2,2-dimethyl-4-oxobutanoic acid

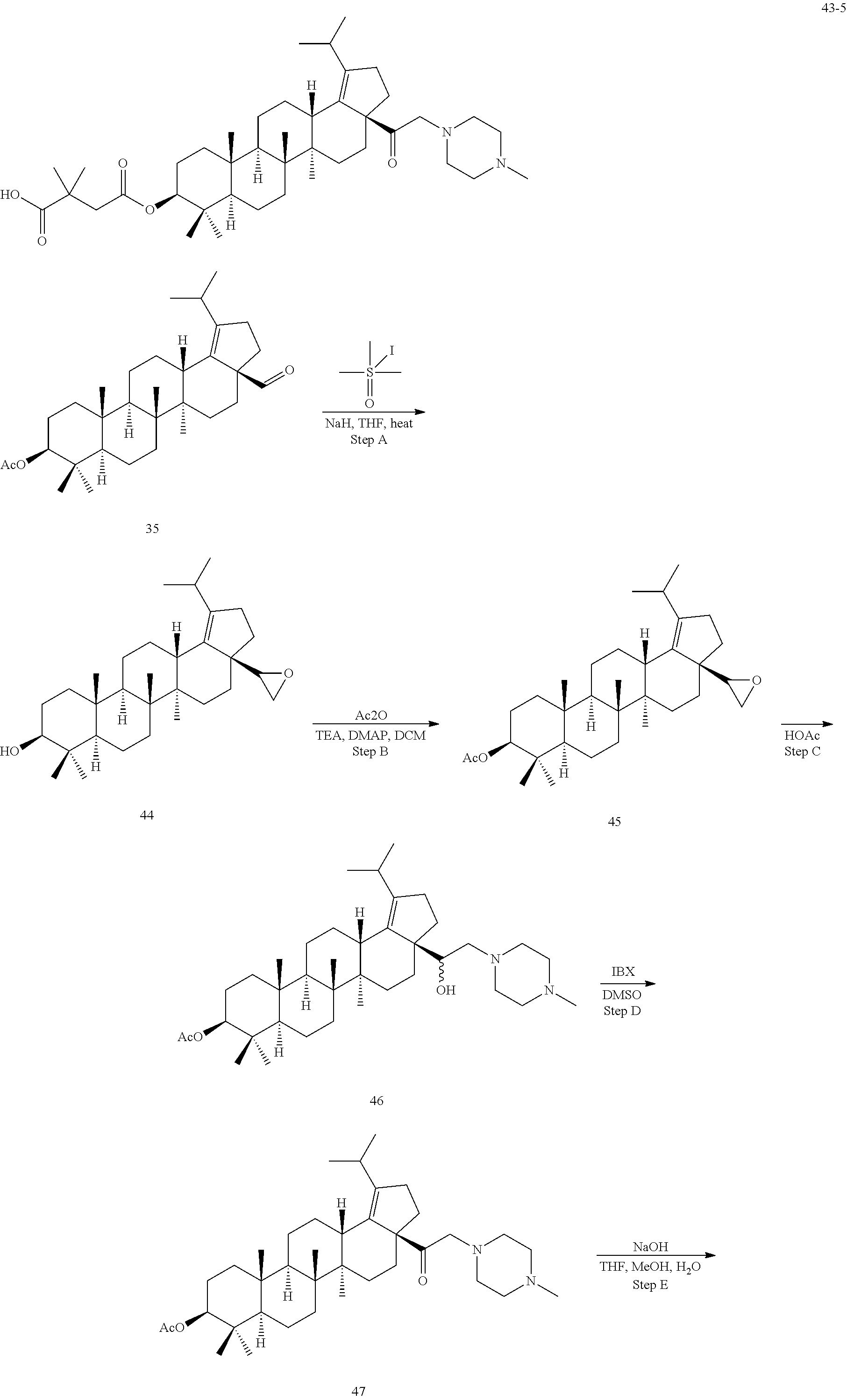

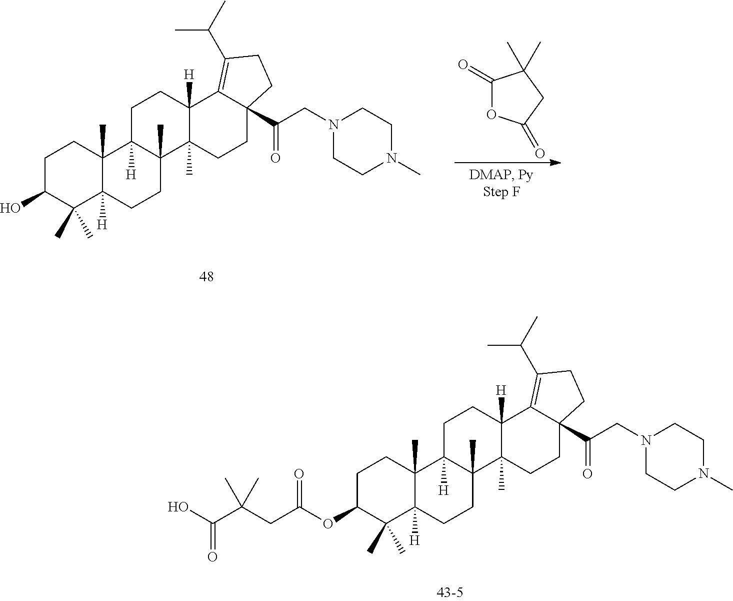

### Step A: Intermediate 44

To a suspension of sodium hydride (60%, 250 mg, 6.25 mmol) in anhydrous THF (50 mL) under an atmosphere of Nitrogen was added trimethylsulfoxonium iodide (900 mg, 4.09 mmol) at room temperature. After the reaction mixture was refluxed for 2 h and cooled down to 60° C., the intermediate 35 (750 mg, 1.554 mmol) in anhydrous THF (2 mL) was added dropwise. The resulting mixture was stirred at 60° C. for 3 h and then at room temperature for 1 h before quenching the reaction with saturated aqueous sodium bicarbonate (50 mL). The reaction mixture was diluted with DCM (200 mL), and the organic phase was washed with water (50 mL×3), saturated brine (50 mL), dried over sodium sulfate, and evaporated in vacuo to give a residue, which was purified by column chromatography on silica gel (Hex:EtOAc=10:1) to afford the intermediate 44 (300 mg, 42.5%) as a white solid.

### Step B: Intermediate 45

To a mixture of the intermediate 44 (300 mg, 0.66 mmol) in DCM (10 mL) were added DMAP (7.25 mg, 0.059 mmol), acetic anhydride (0.182 mL, 1.781 mmol) and Et₃N (0.494 mL, 3.56 mmol). After stirring at room temperature for 1 h, the reaction mixture was diluted with iced water (20 mL), and extracted with DCM (100 mL). The organic phase was washed with water (40 mL×3), brine (40 mL), dried over sodium sulfate, and evaporated in vacuo to give a pale yellow solid. The solid was taken up in MeOH (5 mL), and the precipitates were collected and rinsed with cold MeOH (5 mL) to afford the intermediate 45 (250 mg, 85%) as a white solid.

### Step C: Compound 46

To a solution of the intermediate 45 (250 mg, 0.503 mmol) in 1-methylpiperazine (5 mL) was added acetic acid (0.014 mL, 0.252 mmol). After it was stirred at 150° C. overnight and cooled down to room temperature, the reaction mixture was partitioned between water (50 mL) and DCM (100 mL). The organic phase was then separated, washed with saturated ammonium chloride (40 mL), saturated sodium bicarbonate (40 mL), water (100 mL×3), brine (50 mL), and dried over sodium sulfate. Removal of the solvent gave the intermediate 46 (270 mg, 84%) as a light yellow solid. LC/MS: m/z Calculated 596.5, found 597.4 (M+1)+.

### Step D: Intermediate 47

To a solution of IBX (1.178 g, 4.21 mmol) in DMSO (20 mL) was added the intermediate 46 (270 mg, 0.421 mmol). After it was stirred at 50° C. for 1 h, the reaction mixture was partitioned between water (150 mL) and DCM (50 mL). The organic phase was separated, washed with water (20 mL×3), brine (20 mL), and dried over sodium sulfate. Removal of the solvent gave the intermediate 47 (200 mg, 80%) as a light yellow solid. LC/MS: m/z Calculated 594.5, found 595.4 (M+1)+.

### Step E: Intermediate 48

To a solution of the intermediate 47 (200 mg, 0.336 mmol) in MeOH (3 mL), THF (3 mL), and water (1.5 mL) was added NaOH (1076 mg, 26.9 mmol). After stirring at room temperature for 1 h, the reaction mixture was partitioned between water (150 mL) and DCM (100 mL). The organic phase was separated, washed with water (20 mL×3), brine (20 mL), dried over sodium sulfate, and evaporated in vacuo to give a residue, which was purified by Prep-HPLC to afford the intermediate 48 (77.7 mg, 37.7%) as an off-white solid. LC/MS: m/z Calculated 552.5, found 553.2 (M+1)+.

### Step F: Compound 43-5

To a solution of the intermediate 48 (100 mg, 0.181 mmol) in anhydrous pyridine (3 mL) were added 3,3-dimethyl-dihydrofuran-2,5-dione (232 mg, 1.809 mmol) and DMAP (66.3 mg, 0.543 mmol). After the reaction mixture was heated at 120° C. under microwave for 1.5 h, the solvent was removed in vacuo and the residue was taken up in EtOAc (50 mL). The organic phase was washed with aqueous HCl (0.5 N, 20 mL), water (30 mL×2), brine (30 mL), dried over sodium sulfate, and evaporated to dryness in vacuo to give a residue, which was purified by Prep-HPLC to afford the compound 43-5 (32 mg, 18.74%) as a white solid. ¹H NMR (400 Hz, MeOD) δ ppm 4.51-4.47 (1H, m), 3.75-3.65 (2H, t), 3.32 (4H, br), 3.31-3.27 (1H, m), 3.05 (4H, br), 2.91 (3H, s), 2.67-2.55 (4H, m), 2.32-2.28 (1H, m), 2.20-2.17 (1H, m), 2.06-2.03 (1H, m), 1.95-0.88 (45H, m). LC/MS: m/z Calculated 680.5, found 681.4 (M+1)+.

The compounds in Table 3 were prepared in a manner similar to the indicated procedures above. In each case, X and Y are each CH₂.

TABLE 3

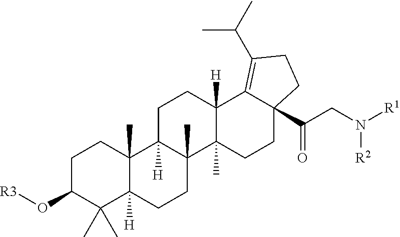

| Example | | | | Synthetic |
|---|---|---|---|---|
| No. | R¹ | R² | R³ | Procedure |
| 17-1 | 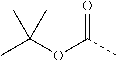 | 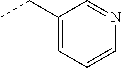 | 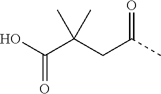 | Ex 13 |
| 17-2 | 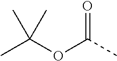 | 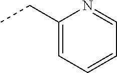 | 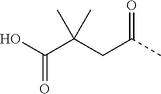 | Ex 13 |
| 17-3 | H | 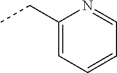 | 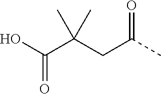 | Ex 13 |
| 17-4 | 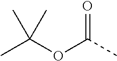 | 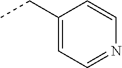 | 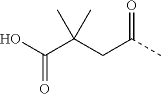 | Ex 13 |
| 17-5 | MeSO₂— | 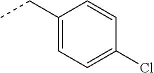 | 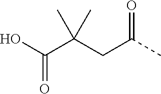 | Ex 13 |
| 17-6 | H | 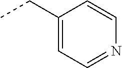 | 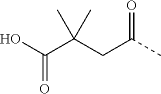 | Ex 13 |
| 17-6 | H | 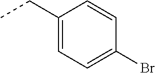 | 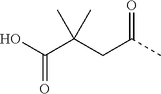 | Ex 13 |
| 17-7 | H | 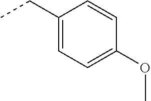 | 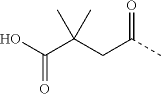 | Ex 13 |
| 17-8 | H | H | 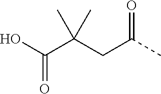 | Ex 13 |
| 17-9 | H | 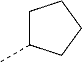 | 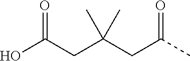 | Ex 13 |
| 17-10 | H | H | 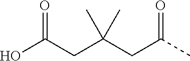 | Ex 13 |
| 17-11 | H | 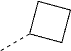 | 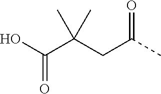 | Ex 13 |
| 17-12 | H | 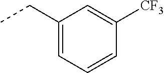 | 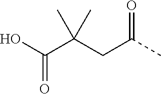 | Ex 13 |
| 17-13 | H | 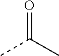 | 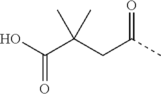 | Ex 13 |
| 17-14 | H | 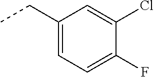 | 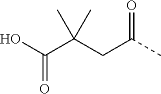 | Ex 13 |
| 17-15 | H | 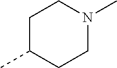 | 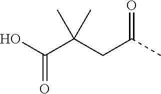 | Ex 13 |
| 17-16 | CH₃ | 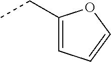 | 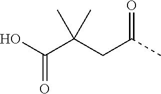 | Ex 13 |
| 17-17 | H | 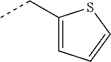 | 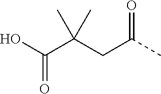 | Ex 13 |
| 17-18 | CH₃ | 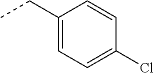 | 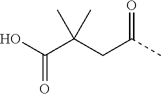 | Ex 13 |
| 17-19 | 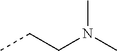 | 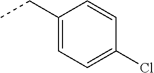 | 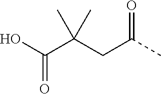 | Ex 17 |
| 17-20 | CH₃ | 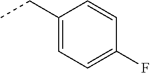 | 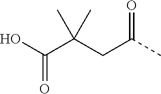 | Ex 13 |
| 17-21 | CH₃ | 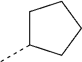 | 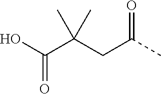 | Ex 13 |
| 17-22 | H | 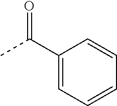 | 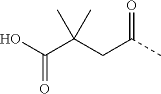 | Ex 13 |
| 17-23 | H | 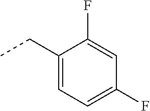 | 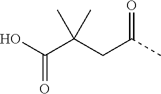 | Ex 13 |
| 17-24 | CH₃ | 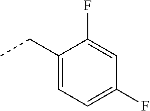 | 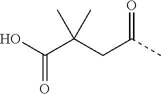 | Ex 13 |
| 17-25 | H | 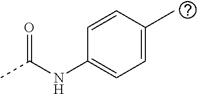 | 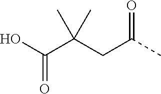 | Ex 13 |
| 17-26 | H | 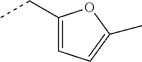 | 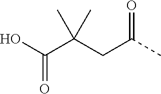 | Ex 13 |
| 17-27 | 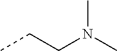 | 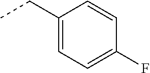 | 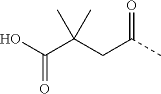 | Ex 13 |
| 17-28 | H | 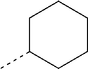 | 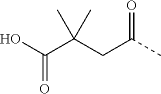 | Ex 13 |
| 17-29 | 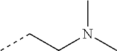 | 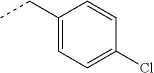 | 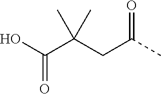 | Ex 13 |
| 17-30 | 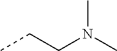 | 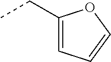 | 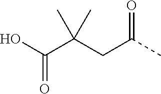 | Ex 13 |

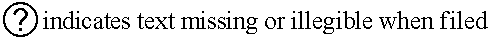

### Example 18

### 4-((3aS,5aR,5bR,7aR,9S,11aR,11bR,13aS)-1-Isopstickyl-5a,5b,8,8,11a-pentamethyl-3a-(2-(4-methylpiperazin-1-yl)-2-oxoacetyl)-3,3a,4,5,5a,5b,6,7,7a,8,9,10,11,11a,11b,12,13,13a-octadecahydro-2H-cyclopenta[a]chrysen-9-yloxy)-2,2-dimethyl-4-oxobutanoic acid

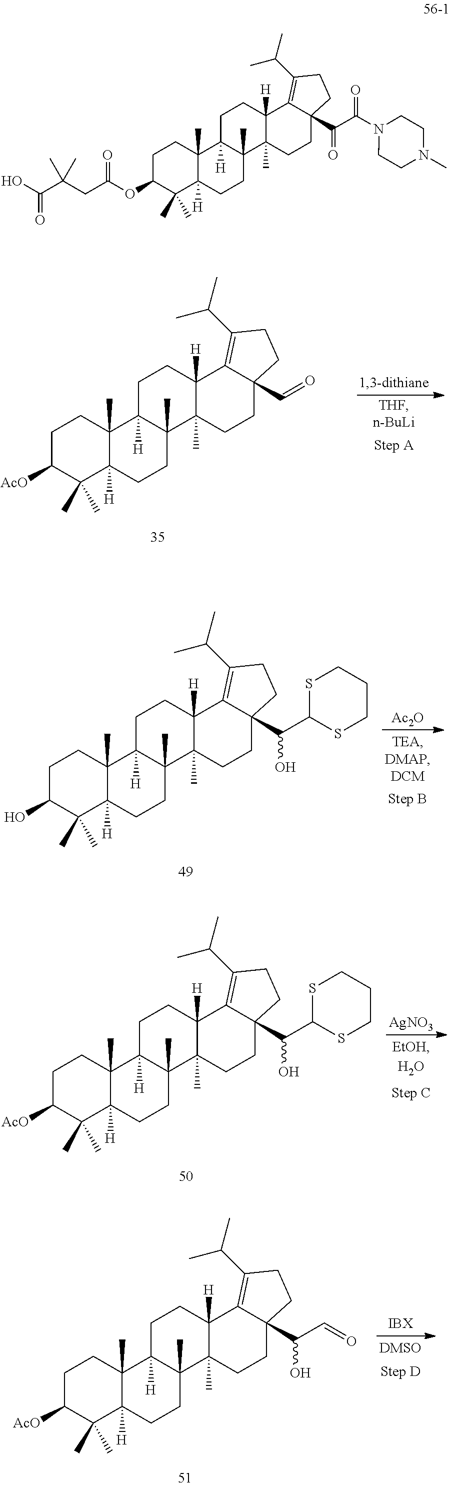

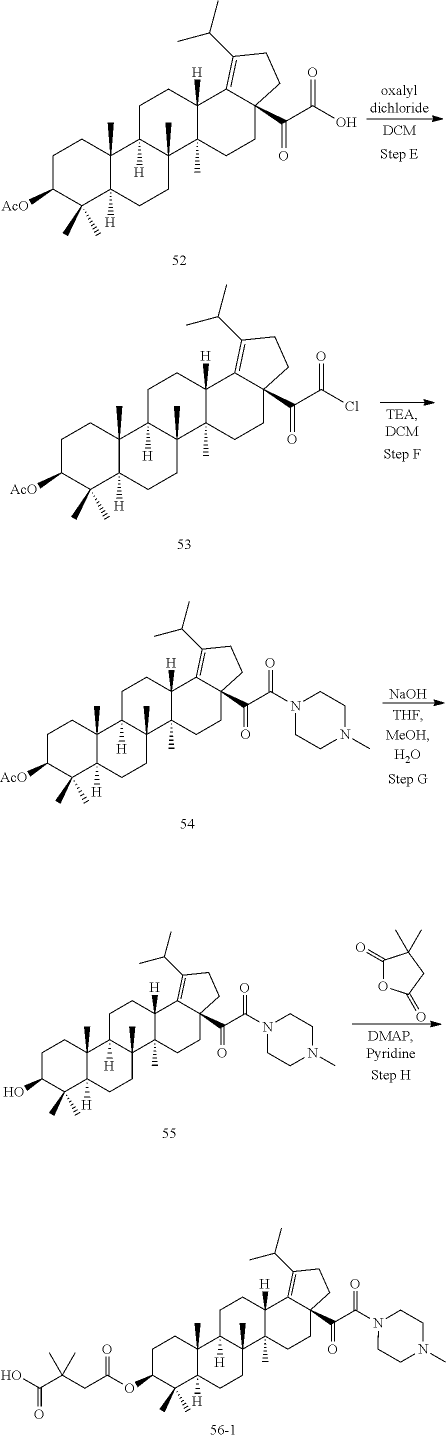

### Step A: Intermediate 49

To a solution of 1,3-dithiane (4 g, 33.2 mmol) in anhydrous THF (50 mL) was added n-BuLi (2.5 M in THF (18 mL, 45 mmol) slowly at −20° C. under an atmosphere of Argon. The resulting mixture was stirred at −20° C. for 2 h. After cooling down to −78° C., the intermediate 35 (3 g, 6.21 mmol) in anhydrous THF (5 mL) was added to the reaction mixture and the resulting mixture was stirred at −78° C. for another 1 h before the reaction was quenched by the addition of 10% solution of sodium bicarbonate (10 mL). The reaction mixture was diluted with DCM (100 mL), and the organic phase was separated, washed with brine, dried over sodium sulfate, and evaporated to dryness in vacuo to give a residue, which was purified by column chromatograph on silica gel (Hex:EtOAc=10:1) to afford the intermediate 49 (1.7 g, 48.8%) as a white solid. LC/MS: m/z calculated 560.3, found 583.3 (M+Na)⁺.

### Step B: Intermediate 50

To a solution of the intermediate 49 (1 g, 1.78 mmol), Et₃N (0.743 mL, 5.35 mmol) and DMAP (65.3 mg, 0.535 mmol) in DCM (100 mL) was added acetic anhydride (0.184 mL, 1.961 mmol). After it was stirred at 0° C. for 4 h, the reaction mixture was partitioned between water (50 mL) and DCM (100 mL). The organic phase was separated, washed with water, brine, and dried over sodium sulfate. Removal of the solvent gave a residue, which was recrystallized from EtOH and DCM to afford the intermediate 50 (510 mg, 47.4%) as a white solid. LC/MS: m/z calculated 602.9, found 625.3 (M+Na)⁺.

### Step C: Intermediate 51

To a solution of the intermediate 50 (2 g, 3.32 mmol) in EtOH (45 mL), and water (5 mL) was added silver nitrate (5.63 g, 33.2 mmol). After the reaction mixture was stirred at 60° C. overnight, the insoluble material was removed by filtration and washed with DCM (100 mL×3). The organic phase was washed with water (100 mL×3), brine (100 mL), and dried over sodium sulfate. Evaporation under reduced pressure gave the intermediate 51 (1.5 g, 88%) as a yellow solid. LC/MS: m/z calculated 512.8, found 535.2 (M+Na)⁺.

### Step D: Intermediate 52

To a solution of the intermediate 51 (700 mg, 1.37 mmol) in DMSO (25 mL) was added IBX (3823 mg, 13.65 mmol). After it was stirred at 30° C. for 1 h, the reaction mixture was partitioned between water (20 mL) and DCM (300 mL). The organic phase was then separated, washed with water, brine, and dried over sodium sulfate. Removal of the solvent gave the intermediate 52 (370 mg, 51.5%) as a yellow solid. LC/MS: m/z calculated 526.3, found 525.3 (M−1)⁻.

### Step E: Intermediate 53

To a solution of the intermediate 52 (260 mg, 0.494 mmol) in DCM (10 mL) was added oxalyl dichloride (0.418 mL, 4.94 mmol). After stirring at room temperature for 1 h, the reaction mixture was evaporated to dryness in vacuo to give the intermediate 53 (269 mg, 100%) as a yellow solid.

### Step F: Intermediate 54

To a solution of the intermediate 53 (270 mg, 0.495 mmol), and Et₃N (551 mg, 5.45 mmol) in DCM (10 mL) was added 1-methylpiperazine (496 mg, 4.95 mmol). After stirring at room temperature for 1 h, the reaction mixture was partitioned between water (20 mL) and DCM (150 mL). The organic phase was separated, washed with water (20 mL), brine (10 mL), dried over sodium sulfate, and evaporated to dryness in vacuo to give the intermediate 54 (220 mg, 73%) as a yellow solid. LC/MS: m/z calculated 608.4, found 609.4 (M+1)⁺.

### Step G: Intermediate 55

To a solution of the intermediate 54 (220 mg, 0.361 mmol) in THF (2 mL), MeOH (2 mL), and water (1 mL) was added NaOH (1.1 g, 28.9 mmol). After stirring at room temperature for 1 h, the reaction mixture was partitioned between water (20 mL) and DCM (150 mL). The organic phase was separated, washed with water (20 mL), brine (10 mL), dried over sodium sulfate, and evaporated to dryness in vacuo to give the intermediate 55 (150 mg, 73.2%) as a yellow solid. LC/MS: m/z calculated 566.4, found 567.3 (M+1)⁺.

### Step H: Compound 56-1

To a solution of the intermediate 55 (678 mg, 5.29 mmol) and DMAP (97 mg, 0.794 mmol) in pyridine (2 mL) was added 3,3-dimethyl-dihydrofuran-2,5-dione (678 mg, 5.29 mmol). After the reaction mixture was stirred at 120° C. for 3 h, the solvent was removed in vacuo and the residue was taken up in EtOAc (50 mL). The organic phase was washed with aqueous HCl (0.5 N, 20 mL), water (30 mL×2), brine (30 mL), dried over sodium sulfate, and evaporated to dryness in vacuo to give a residue, which was purified by Prep-HPLC to afford the title compound 56-1 (30 mg, 14.01%) as a white solid. ¹HNMR (400 MHz, MeOD) δ ppm: 4.51-4.47 (1H, m), 3.72-3.35 (8H, m), 3.29-3.22 (1H, m), 2.98 (3H, s), 2.61 (2H, q, J=16 Hz), 2.50-0.88 (50H, m). LC/MS: calculated 694.5, found 695.4 (M+1)⁺.

### Example 19

### 4-((3aS,5aR,5bR,7aR,9S,11aR,11bR,13aS)-1-Isopstickyl-5a,5b,8,8,11a-pentamethyl-3a-(2-oxo-2-(2-phenylpropan-2-ylamino)acetyl)-3,3a,4,5,5a,5b,6,7,7a,8,9,10,11,11a,11b,12,13,13a-octadecahydro-2H-cyclopenta[a]chrysen-9-yloxy)-2,2-dimethyl-4-oxobutanoic acid

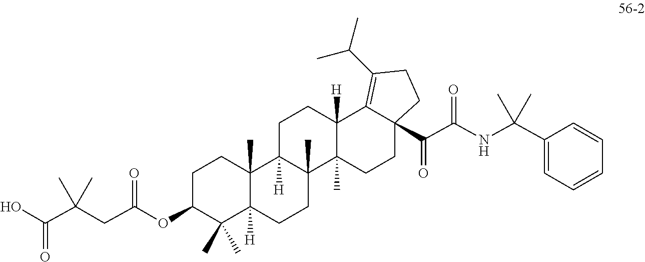

A similar procedure as described in Example 18 was used to prepare the title compound. ¹HNMR (400 MHz, MeOD) δ ppm: 8.23 (1H, s), 7.29-7.28 (2H, m), 7.21-7.17 (2H, m), 7.11-7.07 (1H, m), 4.40-4.36 (1H, m), 3.65-3.44 (1H, m), 3.11-3.04 (1H, m), 2.50 (2H, q, J=16.4 Hz), 2.40-2.05 (6H, m), 1.91-0.77 (49H, m). LC/MS: calculated 729.5, found m/z 730.3 (M+1)+.

### Example 20

### 4-((3aS,5aR,5bR,7aR,9S,11aR,11bR,13aS)-3a-(2-((4-Chlorobenzyl)(methyl)amino)-2-oxoacetyl)-1-isopstickyl-5a,5b,8,8,11a-pentamethyl-3,3a,4,5,5a,5b,6,7,7a,8,9,10,11,11a,11b,12,13,13a-octadecahydro-2H-cyclopenta[a]chrysen-9-yloxy)-2,2-dimethyl-4-oxobutanoic acid

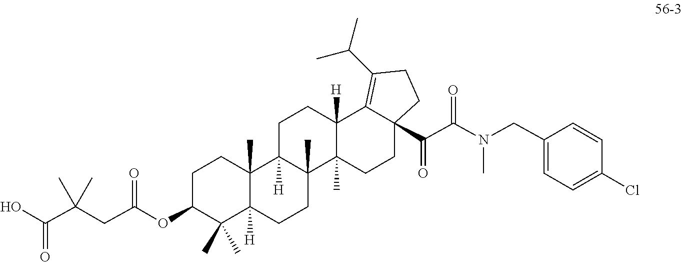

A similar procedure as described in Example 18 was used to prepare the title compound. ¹HNMR (400 MHz, CDCl₃) δ ppm: 7.32-7.25 (3H, m), 7.18 (1H, m), 4.67-4.24 (3H, m), 3.23-3.19 (1H, m), 2.86 (2H, ⅔CH₃, s), 2.79 (1H, ⅓CH₃, s), 2.63 (1H, d, J=15.6 Hz), 2.58 (1H, d, J=15.6 Hz), 2.44-0.79 (50H, m). LC/MS: calculated 749.44, found 772.5 (M+Na)⁺.

1. A compound characterized by the following formula: 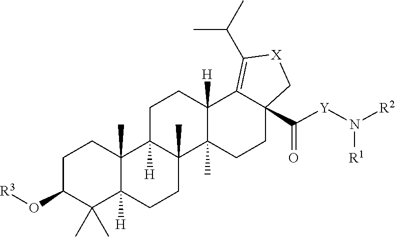
or a pharmaceutically acceptable salt thereof, wherein:
R¹ and R² are each independently H, C₁-C₆-alkyl, t-butyloxycarbonyl, Me-SO₂—, HOOCC(CH₃)₂CH₂C(O)—, CH₃C(O); (R⁴)₂N(CH₂)ₘ—, (R⁵)ₙ-phenyl-Q-, (R⁶)_{q}-Hetaryl-(CH₂)ₚ—, (R⁶)_{q}-Hetalk-(CH₂)ᵣ—, or (R⁶)_{q}-Cycloalk-(CH₂)ₚ—; or R¹ and R², together with the nitrogen atom to which they are attached, form a 3- to 7-membered heterocycloalkyl ring optionally substituted with a methylsulfonyl group or up to two C₁-C₄-alkyl groups;
R³ is 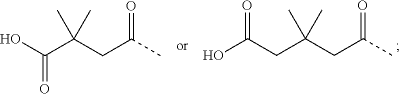
each R⁴ is independently H or C₁-C₆-alkyl;
each R⁵ is independently halo, C₁-C₆-alkyl, C₁-C₆-alkoxy, CF₃, OCF₃, N(CH₃)₂, or NO₂;
each R⁶ is independently halo, C₁-C₆-alkyl, —COOH, —C(O)NH₂, dimethylaminomethyl,
or 1-methyl-4-piperazinylmethyl,
X and Y are each independently methylene or carbonyl;
Q is —(CH₂)ₚ—, —C(O)—, —NH—C(O)—, —CH(CH₃)—, —C(CH₃)₂—, 1,1-cyclopropyldiyl, or 1,1-cyclopentyldiyl;
Hetaryl is a 5-6-membered heteroaryl group;
Hetalk is a 3-7-membered heterocycloalkyl group;
Cycloalk is a 3-6-membered cycloalkyl group;
each m is independently 2 or 3;
each n is independently 0, 1, or 2;
each p is independently 0 or 1;
each q is independently 0, 1, or 2; and
each r is independently 0, 1, 2, 3, or 4.

2. The compound of claim 1 or a pharmaceutically acceptable salt thereof, wherein:
R¹ is H, methyl, dimethylaminoethyl, t-butyloxycarbonyl; Me-SO₂—, or HOOCC(CH₃)₂CH₂C(O)—;
R² is H; (R⁵)ₙ-phenyl-Q-, (R⁶)_{q}-furanyl-(CH₂)ₚ—, (R⁶)_{q}-pyridyl-(CH₂)ₚ—, (R⁶)_{q}-thienyl-(CH₂)ₚ—, 1-methylpyrazol-3-yl, Hetalk-(CH₂)ᵣ—, or C₃-C₆-cycloalkyl-(CH₂)ₚ—, or
R¹ and R², together with the nitrogen atom to which they are attached, form azetidinyl, piperidinyl, morpholino, thiomorpholino, piperazinyl, 4-methyl-piperazin-1-yl, 4-methylsulfonyl-piperazin-1-yl, 2,4-dimethyl-piperazin-1-yl, 4-methyl-diazepan-1-yl, 1-methyl-2-piperazinon-4-yl, thiomorpholine-1,1 dioxide-4-yl; or pyrrolidinyl; and
each R⁵ is independently methyl, methoxy, halo, CF₃, or OCF₃; and
each R⁶ is independently methyl, F, or Cl.

3. The compound of claim 1 or a pharmaceutically acceptable salt thereof, wherein:
X is methylene and Y is methylene;
R¹ is H, methyl, t-butyloxycarbonyl; Me-SO₂—, or dimethylaminoethyl;
R² is H, (R⁵)ₙ-phenyl-(CH₂)ₚ—, (R⁶)ₙ-furanyl-(CH₂)_{q}—, (R⁶)ₙ-pyridyl-(CH₂)_{q}—, (R⁶)_{q}-thienyl-(CH₂)ₚ—, 1-methylpyrazol-3-yl, pyrrolidinyl-(CH₂)ᵣ—, 4-methylpiperazinyl; N-methylpiperidin-4-yl; cyclopropyl-(CH₂)ₚ—, cyclohexyl-(CH₂)ₚ—, or cyclopentyl-(CH₂)ₚ—; or
R¹ and R², together with the nitrogen atom to which they are attached, form azetidinyl, piperazinyl, 4-methyl-piperazin-1-yl, 4-methylsulfonyl-piperazin-1-yl, 2,4-dimethyl-piperazin-1-yl, 4-methyl-diazepan-1-yl, thiomorpholine-1,1 dioxide-4-yl; or pyrrolidinyl;
R⁵ is methyl, methoxy, F, Cl, CF₃, or OCF₃; and
q is 0 or 1.

4. The compound of claim 1 a pharmaceutically acceptable salt thereof, wherein
X is methylene and Y is carbonyl;
R¹ is H, methyl, t-butyloxycarbonyl; Me-SO₂—, or dimethylaminoethyl;
R² is H, (R⁵)ₙ-phenyl-(CH₂)ₚ—, (R⁶)ₙ-furanyl-(CH₂)_{q}—, (R⁶)ₙ-pyridyl-(CH₂)_{q}—, (R⁶)_{q}-thienyl-(CH₂)ₚ—, 1-methylpyrazol-3-yl, pyrrolidinyl-(CH₂)ᵣ—, 4-methylpiperazinyl; N-methylpiperidin-4-yl; cyclopropyl-(CH₂)ₚ—, cyclohexyl-(CH₂)ₚ—, or cyclopentyl-(CH₂)ₚ—; or
R¹ and R², together with the nitrogen atom to which they are attached, form azetidinyl, piperazinyl, 4-methyl-piperazin-1-yl, 4-methylsulfonyl-piperazin-1-yl, 2,4-dimethyl-piperazin-1-yl, 4-methyl-diazepan-1-yl, thiomorpholine-1,1 dioxide-4-yl; or pyrrolidinyl;
R⁵ is methyl, methoxy, F, Cl, CF₃, or OCF₃; and
q is 0 or 1.

5. The compound of claim 1 or a pharmaceutically acceptable salt thereof, wherein
X is carbonyl and Y is carbonyl;
R¹ is H, methyl, t-butyloxycarbonyl; Me-SO₂—, or dimethylaminoethyl;
R² is H, (R⁵)ₙ-phenyl-(CH₂)ₚ—, (R⁶)ₙ-furanyl-(CH₂)_{q}, (R⁶)ₙ-pyridyl-(CH₂)_{q}—, (R⁶)_{q}-thienyl-(CH₂)ₚ—, 1-methylpyrazol-3-yl, pyrrolidinyl-(CH₂)ᵣ—, 4-methylpiperazinyl; N-methylpiperidin-4-yl; cyclopropyl-(CH₂)ₚ—, cyclohexyl-(CH₂)ₚ—, or cyclopentyl-(CH₂)ₚ—; or
R¹ and R², together with the nitrogen atom to which they are attached, form azetidinyl, piperazinyl, 4-methyl-piperazin-1-yl, 4-methylsulfonyl-piperazin-1-yl, 2,4-dimethyl-piperazin-1-yl, 4-methyl-diazepan-1-yl, thiomorpholine-1,1 dioxide-4-yl; or pyrrolidinyl;
R⁵ is methyl, methoxy, F, Cl, CF₃, or OCF₃; and
q is 0 or 1.

6. The compound of claim 1 or a pharmaceutically acceptable salt thereof, wherein
X is carbonyl and Y is methylene;
R¹ is H, methyl, t-butyloxycarbonyl; Me-SO₂—, or dimethylaminoethyl;
R² is H, (R⁵)ₙ-phenyl-(CH₂)ₚ—, (R⁶)ₙ-furanyl-(CH₂)_{q}, (R⁶)ₙ-pyridyl-(CH₂)_{q}—, (R⁶)_{q}-thienyl-(CH₂)ₚ—, 1-methylpyrazol-3-yl, pyrrolidinyl-(CH₂)ᵣ—, 4-methylpiperazinyl; N-methylpiperidin-4-yl; cyclopropyl-(CH₂)ₚ—, cyclohexyl-(CH₂)ₚ—, or cyclopentyl-(CH₂)ₚ—; or
R¹ and R², together with the nitrogen atom to which they are attached, form azetidinyl, piperazinyl, 4-methyl-piperazin-1-yl, 4-methylsulfonyl-piperazin-1-yl, 2,4-dimethyl-piperazin-1-yl, 4-methyl-diazepan-1-yl, thiomorpholine-1,1 dioxide-4-yl; or pyrrolidinyl;
R⁵ is methyl, methoxy, F, Cl, CF₃, or OCF₃; and
q is 0 or 1.

7. The compound of claim 3 or a pharmaceutically acceptable salt thereof, wherein
R¹ is H, CH₃, or dimethylaminoethyl;
R² is (R⁵)ₚ-phenyl-CH₂; thienyl-CH₂—; furanyl-CH₂; pyridinyl-CH₂—; and
R³ is

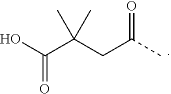

8. The compound of claim 4 or a pharmaceutically acceptable salt thereof, wherein
R¹ is H, CH₃, or dimethylaminoethyl;
R² is (R⁵)ₙ-phenyl-CH₂; thienyl-CH₂—; furanyl-CH₂; pyridinyl-CH₂—; and
R³ is

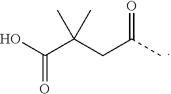

9. The compound of claim 5 or a pharmaceutically acceptable salt thereof, wherein
R¹ is H, CH₃, or dimethylaminoethyl;
R² is (R⁵)ₙ-phenyl-CH₂; thienyl-CH₂—; furanyl-CH₂; pyridinyl-CH₂—; and
R³ is

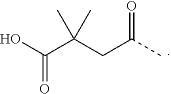

10. The compound of claim 6 or a pharmaceutically acceptable salt thereof, wherein
R¹ is H, CH₃, or dimethylaminoethyl;
R² is (R⁵)ₙ-phenyl-CH₂; thienyl-CH₂—; furanyl-CH₂; pyridinyl-CH₂—; and
R³ is

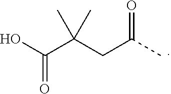

11. A composition comprising a) the compound of claim 1 or a pharmaceutically acceptable salt thereof; and 2) a pharmaceutically acceptable excipient.

12. A method of treating HIV-1 comprising administering to a patient suffering therefrom an effective amount of the compound of claim 1 or a pharmaceutically acceptable salt thereof.
